SEQUENCE LISTING (37 sequences)

SEQ ID NO: 1 - protein, 708 aa, synthetic construct
  REGION 1..708: note = fusion protein comprises a canine insulin leader sequence, a canine proinsulin (K53R, R55K and L86R), a glycine/serine linker, and a canine serum albumin
  REGION 1..24: note = canine insulin leader sequence
  REGION 25..110: note = MISC_FEATURE - modified canine insulin
  REGION 111..124: note = MISC_FEATURE - linker
  REGION 125..708: note = MISC_FEATURE - caninin albumin
MALWMRLLPLLALLALWAPAPTRAFVNQHLCGSHLVEALYLVCGERGFFYTPRAKREVEDLQVRDVELAGAPGEGGLQPLALEGARQKRGIVEQCCTSICSLYQLENYCNGGGGSGGGGSGGGSEAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYSRRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCADDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 2 - DNA, 2127 bp, synthetic construct
  misc_feature 1..2127: note = nucleic acid sequence encoding canine proinsulin-serum albumin fusion
  source 1..2127: mol_type = other DNA
atggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggtagtggtggtggatctgaggcctacaagtccgagatcgcccaccggtacaatgacctgggcgaagaacacttcagaggcctggtgctggtggccttcagccagtacctgcagcagtgccctttcgaggaccacgtgaagctggccaaagaagtgaccgagttcgccaaagcctgtgccgccgaagaaagcggcgccaattgcgacaaaagcctgcacaccctgttcggcgacaagctgtgtacagtggccagcctgagggataagtacggcgacatggccgactgctgcgagaagcaagagcccgacagaaacgagtgctttctggcccacaaggacgacaaccctggcttccctcctctggtggctcctgaacctgatgctctgtgtgccgcctttcaggacaacgagcagctgttcctgggcaagtacctgtacgaaatcgccaggcggcacccctacttttacgcccctgaactgctgtactacgcccagcagtacaagggcgtgttcgccgaatgttgtcaggccgccgataaggccgcttgtctgggaccaaaaatcgaggccctgcgcgagaaggtgctgctgtctagcgccaaagaaaggttcaagtgcgcctctctgcagaagttcggcgatagggccttcaaggcttggagcgtggcaaggctgagccagagattccctaaggccgacttcgccgagatcagcaaggtggtcaccgacctgaccaaggtgcacaaagagtgctgccacggcgacctgctggaatgcgctgatgatagagccgatctggccaagtacatgtgcgagaaccaggacagcatcagcaccaagctgaaagagtgttgcgacaagcccgtgctggaaaagtctcagtgtctggccgaggtggaaagggatgagctgcctggggatctgccttctctggccgctgacttcgtcgaggacaaagaagtctgcaagaactaccaagaggccaaggatgtgtttctgggcaccttcctgtacgagtactccagaaggcaccccgagtacagcgttagcctgctgctgaggctggctaaagagtacgaggccacactggaaaaatgctgcgccaccgacgatcctccaacctgctatgccaaggtgctggacgagttcaagcccctggtggacgagcctcagaacctggtcaagaccaactgcgagctgttcgagaagctgggagagtacggcttccagaacgccctgctcgtgcggtacacaaaaaaggcccctcaggtgtccacacctacactggtcgaggtgtcccggaaactgggaaaagtgggcaccaagtgttgcaagaagcctgagagcgagaggatgtcctgcgccgacgatttcctgagcgtggtgctgaacagactgtgcgtgctgcacgagaaaacccctgtgtctgagcgcgtgaccaagtgctgctctgagagcctggtcaacagacggccttgtttcagcggcctggaagtggacgaaacctacgtgcccaaagagttcaacgccgaaaccttcaccttccacgccgacctgtgtaccctgcctgaggccgagaaacaagtgaagaaacagacagccctggtcgagctgctgaagcacaagcctaaagccaccgatgagcagctgaaaaccgtgatgggagacttcggcgccttcgtggaaaagtgttgtgccgcagagaacaaagagggatgcttcagcgaagagggccccaagctggttgctgctgctcaagctgccctggtgtaa

SEQ ID NO: 3 - protein, 809 aa, synthetic construct
  REGION 1..809: note = fusion protein comprises a canine insulin leader sequence and a canine proinsulin (K53R, R55K and L86R), , a glycine/serine linker, and a canine transferrin
  REGION 1..14: note = Signal
  REGION 15..110: note = MISC_FEATURE - modified canine insulin
  REGION 111..124: note = MISC_FEATURE - linker
  REGION 125..809: note = MISC_FEATURE - canine transferrin
MALWMRLLPLLALLALWAPAPTRAFVNQHLCGSHLVEALYLVCGERGFFYTPRAKREVEDLQVRDVELAGAPGEGGLQPLALEGARQKRGIVEQCCTSICSLYQLENYCNGGGGSGGGGSGGGSVSPEKTVRWCTVSNHEASKCSSFMENMKTVLENGPFVSCVKRTSYLECIKAIWANEADAVTLDAGLVFEAGLNPYNLKPVVAEFYGKDKQTRYYAVAVVKKSSTFNLNQLQGKKSCHTGLGRSAGWNIPMGLLYWKLPEPRESLQKAASSFFTASCVPCVDRTAFPKLCQLCVGKGTDKCACSNHEPYFGYSGAFKCLMEDAGEVAFVKHSTVLENLPNKADLDQYELLCPDNKRKPVGEYKQCYLAKVPSHAVVARSVGGKEDLIWELLNQAQEHYGKDKSKVFQLFSSTLGKDLLFKDSAEGFFRIPPKMDTWLYLGYEYVTALRNLREDVRPDTPRDECKKVKWCAVGHHEIAKCDEWSVNSEGKIECESAESTEDCIAKIAKGEADAMSLDGGYIYIAGQCGLVPVLAENYKTQGSTCSNTAEEGYLAVAVVKRLDKTISWNNLQGRKSCHTAVDRTAGWNIPMGLLYNRINHCEDKFFSQGCAPGSMRNSSLCALCIGSANVPGKECVPNNHERYYGYTGAFRCLVEKGDVAFVKDQTVLQNTGGKNTEDWAKDLKEEDFELLCPDGQRKSVDKAPECFLAKAPNHAVVSRKDKASCVSKMLLDQQLLFGRNGNDCSGKFCLFHSATKDLLFRDDTQCLAKLPEDTTYKSYLGAEYITAVANLRQCSTSKLLEACTFHTS

SEQ ID NO: 4 - DNA, 2430 bp, synthetic construct
  misc_feature 1..2430: note = Coding sequence for fusion protein comprises a canine insulin leader sequence and a canine proinsulin (K53R, R55K and L86R), , a glycine/serine linker, and a canine transferrin
  source 1..2430: mol_type = other DNA
atggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggttcaggtggtggatctgtgtctcccgaaaagaccgtgcggtggtgcaccgtgtctaatcacgaggccagcaagtgcagctccttcatggaaaacatgaagaccgtcctggaaaacggccccttcgtgtcctgcgtgaagaggaccagctacctggaatgcatcaaggccatctgggccaacgaggccgatgccgttacactggatgccggactggtgtttgaggccggactgaacccctacaacctgaagcctgtggtggccgagttctacggcaaggataagcagacccggtactacgccgtggccgtggtcaagaagtccagcaccttcaacctgaatcagctgcagggcaagaagtcttgccacaccggactgggaagaagcgccggctggaatatccctatgggcctgctgtactggaagctgcccgagcctagagagtctctgcagaaggccgccagcagcttcttcaccgctagctgtgtgccttgcgtggacaggaccgcctttcctaagctgtgccagctgtgtgtcggcaagggcaccgataagtgcgcctgcagcaatcacgagccctacttcggatacagcggcgccttcaagtgcctgatggaagatgctggcgaggtggcctttgtgaagcactccacagtgctggaaaatctgcccaacaaggccgacctggaccagtacgaactgctgtgccccgacaacaagagaaagcccgtgggcgagtacaagcagtgctacctggccaaagtgcctagccatgccgtggtggctagaagcgtcggcggaaaagaggacctgatctgggagctgctgaatcaggcccaagagcactacggaaaggacaagagcaaggtgttccagctgttcagcagcaccctgggcaaagacctgctgttcaaggatagcgccgagggcttcttcaggatccctcctaagatggacacctggctgtacctgggctacgagtacgtgacagccctgaggaacctgcgggaagatgtgcggcctgacacacccagggacgagtgcaagaaagtgaagtggtgcgccgtgggccaccacgagatcgccaaatgtgatgagtggagcgtgaacagcgagggcaagatcgagtgcgagtctgccgagtctaccgaggactgcattgccaaaatcgccaaaggcgaggccgacgccatgtctctggatggcggctacatctatatcgccggccagtgtggactggtgcctgtgctggctgagaactacaagacccagggctccacctgtagcaacaccgccgaagagggatatctggccgtggctgtcgtgaagagactggacaagaccatctcctggaacaacctgcaggggcgcaagagctgtcacaccgccgttgatagaaccgccggatggaacattccaatgggactcctctacaaccggatcaaccactgcgaggacaagttctttagccagggatgtgcccctggcagcatgaggaatagtagcctgtgcgccctgtgcatcggcagcgctaatgtgcctggcaaagaatgcgtgccaaacaaccacgagaggtactacggctacaccggggccttcagatgtctggtcgaaaaaggcgacgtggcattcgtgaaggaccagaccgtgctgcagaataccggcggcaagaacaccgaggattgggccaaagacctcaaagaagaggacttcgagctgctgtgtcctgacggccagagaaagagcgtggacaaggcccctgagtgctttctggccaaggctcctaatcacgccgtggtgtctcggaaggataaggcctcttgcgtgtccaagatgctgctcgaccagcaactgctgttcggcagaaacggcaatgactgcagcggcaagttctgcctgttccacagcgccaccaaggatctgctgttccgggacgatacacagtgcctggctaagctgcctgaggacaccacctacaagtcttacctgggagccgagtacatcacagccgtggccaatctgagacagtgcagcacctccaaactgctggaagcctgcaccttccacaccagctga

SEQ ID NO: 5 - protein, 110 aa, synthetic construct
  REGION 1..110: note = a canine insulin leader sequence and a canine proinsulin (K53R, R55K and L86R with no fusoin
  SIGNAL 1..24: note = canine insulin leader
  REGION 25..110: note = cINS-2-1 Peptide with modified cleavage site
MALWMRLLPLLALLALWAPAPTRAFVNQHLCGSHLVEALYLVCGERGFFYTPRAKREVEDLQVRDVELAGAPGEGGLQPLALEGARQKRGIVEQCCTSICSLYQLENYCN

SEQ ID NO: 6 - DNA, 336 bp, synthetic construct
  misc_feature 1..336: note = canine insulin leader sequence and a canine proinsulin (K53R, R55K and L86R) coding sequence
  source 1..336: mol_type = other DNA
atggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctcgggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatga

SEQ ID NO: 7 - protein, 24 aa, Canis lupus
MALWMRLLPLLALLALWAPAPTRA

SEQ ID NO: 8 - protein, 14 aa, synthetic construct
  REGION 1..14: note = Linker 1
GGGGSGGGGSGGGS

SEQ ID NO: 9 - protein, 24 aa, Felis catus
MAPWTRLLPLLALLSLWIPAPTRA

SEQ ID NO: 10 - protein, 86 aa, Canis lupus
FVNQHLCGSHLVEALYLVCGERGFFYTPKARRKREDLQVRDVELAGAPGEGGLQPLALEGALRKRGIVEQCCTSICSLYQLENYCN

SEQ ID NO: 11 - DNA, 336 bp, Canis lupus
  source 1..336: mol_type = unassigned DNA
atggccctgtggatgaggctgctgcctctgctggccctgctggccctgtgggcccctgcccctaccagggccttcgtgaaccagcacctgtgcggcagccacctggtggaggccctgtacctggtgtgcggcgagaggggcttcttctacacccctaaggccaggaggaagagggaggacctgcaggtgagggacgtggagctggccggcgcccctggcgagggcggcctgcagcctctggccctggagggcgccaggaggaagaggggcatcgtggagcagtgctgcaccagcatctgcagcctgtaccagctggagaactactgcaactgatga

SEQ ID NO: 12 - protein, 20 aa, Canis lupus
MYKMQLLSCIALTLVLVANS

SEQ ID NO: 13 - protein, 20 aa, Felis catus
MYKIQLLSCIALTLILVTNS

SEQ ID NO: 14 - protein, 90 aa, synthetic construct
  REGION 1..90: note = Mutant Canine Proinsulin
PTRAFVNQHLCGSHLVEALYLVCGERGFFYTPRAKREVEDLQVRDVELAGAPGEGGLQPLALEGARQKRGIVEQCCTSICSLYQLENYCN

SEQ ID NO: 15 - DNA, 264 bp, synthetic construct
  misc_feature 1..264: note = mutant canine proinsulin w/o leader or fusion coding sequence
  source 1..264: mol_type = other DNA
ttcgtgaaccagcacctgtgcggctctcatctggtggaagccctgtatctcgtgtgcggcgagaggggctttttctacacccctcgggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatga

SEQ ID NO: 16 - protein, 584 aa, Canis lupus
EAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYSRRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCADDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 17 - protein, 685 aa, Canis lupus
VSPEKTVRWCTVSNHEASKCSSFMENMKTVLENGPFVSCVKRTSYLECIKAIWANEADAVTLDAGLVFEAGLNPYNLKPVVAEFYGKDKQTRYYAVAVVKKSSTFNLNQLQGKKSCHTGLGRSAGWNIPMGLLYWKLPEPRESLQKAASSFFTASCVPCVDRTAFPKLCQLCVGKGTDKCACSNHEPYFGYSGAFKCLMEDAGEVAFVKHSTVLENLPNKADLDQYELLCPDNKRKPVGEYKQCYLAKVPSHAVVARSVGGKEDLIWELLNQAQEHYGKDKSKVFQLFSSTLGKDLLFKDSAEGFFRIPPKMDTWLYLGYEYVTALRNLREDVRPDTPRDECKKVKWCAVGHHEIAKCDEWSVNSEGKIECESAESTEDCIAKIAKGEADAMSLDGGYIYIAGQCGLVPVLAENYKTQGSTCSNTAEEGYLAVAVVKRLDKTISWNNLQGRKSCHTAVDRTAGWNIPMGLLYNRINHCEDKFFSQGCAPGSMRNSSLCALCIGSANVPGKECVPNNHERYYGYTGAFRCLVEKGDVAFVKDQTVLQNTGGKNTEDWAKDLKEEDFELLCPDGQRKSVDKAPECFLAKAPNHAVVSRKDKASCVSKMLLDQQLLFGRNGNDCSGKFCLFHSATKDLLFRDDTQCLAKLPEDTTYKSYLGAEYITAVANLRQCSTSKLLEACTFHTS

SEQ ID NO: 18 - protein, 608 aa, Felis catus
MKWVTFISLLLLFSSAYSRGVTRREAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 19 - DNA, 15 bp, synthetic construct
  misc_feature 1..15: note = Linker 2
  source 1..15: mol_type = other DNA
ggggsggggsggggs

SEQ ID NO: 20 - DNA, 127 bp, synthetic construct
  misc_feature 1..127: note = Rabbit Beta Globin polyA
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 21 - DNA, 2211 bp, synthetic construct
  misc_feature 1..2211: note = AAVrh91 VP1 coding sequence
  source 1..2211: mol_type = other DNA
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 22 - protein, 736 aa, synthetic construct
  REGION 1..736: note = AAVrh91 encoded amino acid sequence
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 23 - DNA, 2211 bp, synthetic construct
  misc_feature 1..2211: note = AAVrh91 VP1 engineered coding sequence
  source 1..2211: mol_type = other DNA
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 24 - protein, 86 aa, Felis catus
FVNQHLCGSHLVEALYLVCGERGFFYTPKARREAEDLQGKDAELGEAPGAGGLQPSALEAPLQKRGIVEQCCASVCSLYQLEHYCN

SEQ ID NO: 25 - protein, 86 aa, synthetic construct
  REGION 1..86: note = Mutant feline insulin (no leader or fusion)
FVNQHLCGSHLVEALYLVCGERGFFYTPRAKREAEDLQGKDAELGEAPGAGGLQPSALEAPRQKRGIVEQCCASVCSLYQLEHYCN

SEQ ID NO: 26 - DNA, 4095 bp, synthetic construct
  misc_feature 1..4095: note = CB7.CI.cINS2-1_3xGSsp.ALB.RBG
  misc_feature 1..382: note = CMV IE promoter
  misc_feature 1..666: note = CB7 promoter element
  misc_feature 385..666: note = CB promoter
  misc_feature 639..642: note = Tata
  misc_feature 759..1731: note = Tata
  misc_feature 1794..3936: note = canine preinsulin
  misc_feature 1800..1808: note = canine preinsulin
  misc_feature 1806..3936: note = canine preinsulin
  misc_feature 3969..4095: note = canine preinsulin
  misc_feature 3969..4095: note = canine preinsulin fusion with canine albumin
  source 1..4095: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgtttaaacttaagcttggtaccgagctcggatccactagtccagtgtggtggaattcgccaccatggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggtagtggtggtggatctgaggcctacaagtccgagatcgcccaccggtacaatgacctgggcgaagaacacttcagaggcctggtgctggtggccttcagccagtacctgcagcagtgccctttcgaggaccacgtgaagctggccaaagaagtgaccgagttcgccaaagcctgtgccgccgaagaaagcggcgccaattgcgacaaaagcctgcacaccctgttcggcgacaagctgtgtacagtggccagcctgagggataagtacggcgacatggccgactgctgcgagaagcaagagcccgacagaaacgagtgctttctggcccacaaggacgacaaccctggcttccctcctctggtggctcctgaacctgatgctctgtgtgccgcctttcaggacaacgagcagctgttcctgggcaagtacctgtacgaaatcgccaggcggcacccctacttttacgcccctgaactgctgtactacgcccagcagtacaagggcgtgttcgccgaatgttgtcaggccgccgataaggccgcttgtctgggaccaaaaatcgaggccctgcgcgagaaggtgctgctgtctagcgccaaagaaaggttcaagtgcgcctctctgcagaagttcggcgatagggccttcaaggcttggagcgtggcaaggctgagccagagattccctaaggccgacttcgccgagatcagcaaggtggtcaccgacctgaccaaggtgcacaaagagtgctgccacggcgacctgctggaatgcgctgatgatagagccgatctggccaagtacatgtgcgagaaccaggacagcatcagcaccaagctgaaagagtgttgcgacaagcccgtgctggaaaagtctcagtgtctggccgaggtggaaagggatgagctgcctggggatctgccttctctggccgctgacttcgtcgaggacaaagaagtctgcaagaactaccaagaggccaaggatgtgtttctgggcaccttcctgtacgagtactccagaaggcaccccgagtacagcgttagcctgctgctgaggctggctaaagagtacgaggccacactggaaaaatgctgcgccaccgacgatcctccaacctgctatgccaaggtgctggacgagttcaagcccctggtggacgagcctcagaacctggtcaagaccaactgcgagctgttcgagaagctgggagagtacggcttccagaacgccctgctcgtgcggtacacaaaaaaggcccctcaggtgtccacacctacactggtcgaggtgtcccggaaactgggaaaagtgggcaccaagtgttgcaagaagcctgagagcgagaggatgtcctgcgccgacgatttcctgagcgtggtgctgaacagactgtgcgtgctgcacgagaaaacccctgtgtctgagcgcgtgaccaagtgctgctctgagagcctggtcaacagacggccttgtttcagcggcctggaagtggacgaaacctacgtgcccaaagagttcaacgccgaaaccttcaccttccacgccgacctgtgtaccctgcctgaggccgagaaacaagtgaagaaacagacagccctggtcgagctgctgaagcacaagcctaaagccaccgatgagcagctgaaaaccgtgatgggagacttcggcgccttcgtggaaaagtgttgtgccgcagagaacaaagagggatgcttcagcgaagagggccccaagctggttgctgctgctcaagctgccctggtgtaatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 27 - DNA, 4510 bp, synthetic construct
  misc_feature 1..4510: note = ITR.CB7.CI.cINS2-1.3xGSsp.ALB.RBG
  misc_feature 1..130: note = 5' - AAV2 ITR
  misc_feature 198..579: note = CMV IE promoter
  misc_feature 198..863: note = CB7 promoter element
  misc_feature 582..863: note = CB promoter
  misc_feature 836..839: note = Tata
  misc_feature 956..1928: note = chicken beta-actin intron
  misc_feature 1929..1934: note = mutant canine NGFV41Vk4
  misc_feature 1991..4133: note = canine pre-proinsulin
  misc_feature 1991..4133: note = canine pre-proinsulin fusion witth canine albumin
  misc_feature 1997..2005: note = Kozak
  misc_feature 4166..4292: note = rabbit globin polyA
  misc_feature 4381..4510: note = 3' AAV2-ITR
  source 1..4510: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgtttaaacttaagcttggtaccgagctcggatccactagtccagtgtggtggaattcgccaccatggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggtagtggtggtggatctgaggcctacaagtccgagatcgcccaccggtacaatgacctgggcgaagaacacttcagaggcctggtgctggtggccttcagccagtacctgcagcagtgccctttcgaggaccacgtgaagctggccaaagaagtgaccgagttcgccaaagcctgtgccgccgaagaaagcggcgccaattgcgacaaaagcctgcacaccctgttcggcgacaagctgtgtacagtggccagcctgagggataagtacggcgacatggccgactgctgcgagaagcaagagcccgacagaaacgagtgctttctggcccacaaggacgacaaccctggcttccctcctctggtggctcctgaacctgatgctctgtgtgccgcctttcaggacaacgagcagctgttcctgggcaagtacctgtacgaaatcgccaggcggcacccctacttttacgcccctgaactgctgtactacgcccagcagtacaagggcgtgttcgccgaatgttgtcaggccgccgataaggccgcttgtctgggaccaaaaatcgaggccctgcgcgagaaggtgctgctgtctagcgccaaagaaaggttcaagtgcgcctctctgcagaagttcggcgatagggccttcaaggcttggagcgtggcaaggctgagccagagattccctaaggccgacttcgccgagatcagcaaggtggtcaccgacctgaccaaggtgcacaaagagtgctgccacggcgacctgctggaatgcgctgatgatagagccgatctggccaagtacatgtgcgagaaccaggacagcatcagcaccaagctgaaagagtgttgcgacaagcccgtgctggaaaagtctcagtgtctggccgaggtggaaagggatgagctgcctggggatctgccttctctggccgctgacttcgtcgaggacaaagaagtctgcaagaactaccaagaggccaaggatgtgtttctgggcaccttcctgtacgagtactccagaaggcaccccgagtacagcgttagcctgctgctgaggctggctaaagagtacgaggccacactggaaaaatgctgcgccaccgacgatcctccaacctgctatgccaaggtgctggacgagttcaagcccctggtggacgagcctcagaacctggtcaagaccaactgcgagctgttcgagaagctgggagagtacggcttccagaacgccctgctcgtgcggtacacaaaaaaggcccctcaggtgtccacacctacactggtcgaggtgtcccggaaactgggaaaagtgggcaccaagtgttgcaagaagcctgagagcgagaggatgtcctgcgccgacgatttcctgagcgtggtgctgaacagactgtgcgtgctgcacgagaaaacccctgtgtctgagcgcgtgaccaagtgctgctctgagagcctggtcaacagacggccttgtttcagcggcctggaagtggacgaaacctacgtgcccaaagagttcaacgccgaaaccttcaccttccacgccgacctgtgtaccctgcctgaggccgagaaacaagtgaagaaacagacagccctggtcgagctgctgaagcacaagcctaaagccaccgatgagcagctgaaaaccgtgatgggagacttcggcgccttcgtggaaaagtgttgtgccgcagagaacaaagagggatgcttcagcgaagagggccccaagctggttgctgctgctcaagctgccctggtgtaatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 4335 bp, synthetic construct
  misc_feature 1..4335: note = CB7.CI.cINS2-1.3xGSsp.TF.rBG
  misc_feature 1..382: note = CMV IE promoter
  misc_feature 1..666: note = CB7 promoter element
  misc_feature 385..666: note = CB promoter
  misc_feature 639..642: note = chicken beta-actin intron
  misc_feature 1743..4176: note = canine preinsulin fusion with transferrin
  misc_feature 4209..4335: note = rabbit globin polyA
  source 1..4335: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggttcaggtggtggatctgtgtctcccgaaaagaccgtgcggtggtgcaccgtgtctaatcacgaggccagcaagtgcagctccttcatggaaaacatgaagaccgtcctggaaaacggccccttcgtgtcctgcgtgaagaggaccagctacctggaatgcatcaaggccatctgggccaacgaggccgatgccgttacactggatgccggactggtgtttgaggccggactgaacccctacaacctgaagcctgtggtggccgagttctacggcaaggataagcagacccggtactacgccgtggccgtggtcaagaagtccagcaccttcaacctgaatcagctgcagggcaagaagtcttgccacaccggactgggaagaagcgccggctggaatatccctatgggcctgctgtactggaagctgcccgagcctagagagtctctgcagaaggccgccagcagcttcttcaccgctagctgtgtgccttgcgtggacaggaccgcctttcctaagctgtgccagctgtgtgtcggcaagggcaccgataagtgcgcctgcagcaatcacgagccctacttcggatacagcggcgccttcaagtgcctgatggaagatgctggcgaggtggcctttgtgaagcactccacagtgctggaaaatctgcccaacaaggccgacctggaccagtacgaactgctgtgccccgacaacaagagaaagcccgtgggcgagtacaagcagtgctacctggccaaagtgcctagccatgccgtggtggctagaagcgtcggcggaaaagaggacctgatctgggagctgctgaatcaggcccaagagcactacggaaaggacaagagcaaggtgttccagctgttcagcagcaccctgggcaaagacctgctgttcaaggatagcgccgagggcttcttcaggatccctcctaagatggacacctggctgtacctgggctacgagtacgtgacagccctgaggaacctgcgggaagatgtgcggcctgacacacccagggacgagtgcaagaaagtgaagtggtgcgccgtgggccaccacgagatcgccaaatgtgatgagtggagcgtgaacagcgagggcaagatcgagtgcgagtctgccgagtctaccgaggactgcattgccaaaatcgccaaaggcgaggccgacgccatgtctctggatggcggctacatctatatcgccggccagtgtggactggtgcctgtgctggctgagaactacaagacccagggctccacctgtagcaacaccgccgaagagggatatctggccgtggctgtcgtgaagagactggacaagaccatctcctggaacaacctgcaggggcgcaagagctgtcacaccgccgttgatagaaccgccggatggaacattccaatgggactcctctacaaccggatcaaccactgcgaggacaagttctttagccagggatgtgcccctggcagcatgaggaatagtagcctgtgcgccctgtgcatcggcagcgctaatgtgcctggcaaagaatgcgtgccaaacaaccacgagaggtactacggctacaccggggccttcagatgtctggtcgaaaaaggcgacgtggcattcgtgaaggaccagaccgtgctgcagaataccggcggcaagaacaccgaggattgggccaaagacctcaaagaagaggacttcgagctgctgtgtcctgacggccagagaaagagcgtggacaaggcccctgagtgctttctggccaaggctcctaatcacgccgtggtgtctcggaaggataaggcctcttgcgtgtccaagatgctgctcgaccagcaactgctgttcggcagaaacggcaatgactgcagcggcaagttctgcctgttccacagcgccaccaaggatctgctgttccgggacgatacacagtgcctggctaagctgcctgaggacaccacctacaagtcttacctgggagccgagtacatcacagccgtggccaatctgagacagtgcagcacctccaaactgctggaagcctgcaccttccacaccagctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 29 - DNA, 4750 bp, synthetic construct
  misc_feature 1..4750: note = CB7.CI.cINS2-1.3xGS.TF.rBG.ITR
  misc_feature 1..130: note = AAV2 - shortened 5' ITR
  misc_feature 198..579: note = CMV IE promoter
  misc_feature 582..863: note = CB promoter
  misc_feature 836..839: note = TATA promoter
  misc_feature 956..1928: note = chicken beta-actin intron
  misc_feature 1940..4373: note = chicken beta-actin intron
  misc_feature 4406..4532: note = rabbit globin polyA
  misc_feature 4621..4750: note = rabbit globin polyA
  source 1..4750: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggttcaggtggtggatctgtgtctcccgaaaagaccgtgcggtggtgcaccgtgtctaatcacgaggccagcaagtgcagctccttcatggaaaacatgaagaccgtcctggaaaacggccccttcgtgtcctgcgtgaagaggaccagctacctggaatgcatcaaggccatctgggccaacgaggccgatgccgttacactggatgccggactggtgtttgaggccggactgaacccctacaacctgaagcctgtggtggccgagttctacggcaaggataagcagacccggtactacgccgtggccgtggtcaagaagtccagcaccttcaacctgaatcagctgcagggcaagaagtcttgccacaccggactgggaagaagcgccggctggaatatccctatgggcctgctgtactggaagctgcccgagcctagagagtctctgcagaaggccgccagcagcttcttcaccgctagctgtgtgccttgcgtggacaggaccgcctttcctaagctgtgccagctgtgtgtcggcaagggcaccgataagtgcgcctgcagcaatcacgagccctacttcggatacagcggcgccttcaagtgcctgatggaagatgctggcgaggtggcctttgtgaagcactccacagtgctggaaaatctgcccaacaaggccgacctggaccagtacgaactgctgtgccccgacaacaagagaaagcccgtgggcgagtacaagcagtgctacctggccaaagtgcctagccatgccgtggtggctagaagcgtcggcggaaaagaggacctgatctgggagctgctgaatcaggcccaagagcactacggaaaggacaagagcaaggtgttccagctgttcagcagcaccctgggcaaagacctgctgttcaaggatagcgccgagggcttcttcaggatccctcctaagatggacacctggctgtacctgggctacgagtacgtgacagccctgaggaacctgcgggaagatgtgcggcctgacacacccagggacgagtgcaagaaagtgaagtggtgcgccgtgggccaccacgagatcgccaaatgtgatgagtggagcgtgaacagcgagggcaagatcgagtgcgagtctgccgagtctaccgaggactgcattgccaaaatcgccaaaggcgaggccgacgccatgtctctggatggcggctacatctatatcgccggccagtgtggactggtgcctgtgctggctgagaactacaagacccagggctccacctgtagcaacaccgccgaagagggatatctggccgtggctgtcgtgaagagactggacaagaccatctcctggaacaacctgcaggggcgcaagagctgtcacaccgccgttgatagaaccgccggatggaacattccaatgggactcctctacaaccggatcaaccactgcgaggacaagttctttagccagggatgtgcccctggcagcatgaggaatagtagcctgtgcgccctgtgcatcggcagcgctaatgtgcctggcaaagaatgcgtgccaaacaaccacgagaggtactacggctacaccggggccttcagatgtctggtcgaaaaaggcgacgtggcattcgtgaaggaccagaccgtgctgcagaataccggcggcaagaacaccgaggattgggccaaagacctcaaagaagaggacttcgagctgctgtgtcctgacggccagagaaagagcgtggacaaggcccctgagtgctttctggccaaggctcctaatcacgccgtggtgtctcggaaggataaggcctcttgcgtgtccaagatgctgctcgaccagcaactgctgttcggcagaaacggcaatgactgcagcggcaagttctgcctgttccacagcgccaccaaggatctgctgttccgggacgatacacagtgcctggctaagctgcctgaggacaccacctacaagtcttacctgggagccgagtacatcacagccgtggccaatctgagacagtgcagcacctccaaactgctggaagcctgcaccttccacaccagctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - DNA, 2262 bp, synthetic construct
  misc_feature 1..2262: note = CB7.CI.cINS2-1(N)-rBG
  misc_feature 1..382: note = CMV IE promoter
  misc_feature 1..666: note = CB7 promoter
  misc_feature 385..666: note = CB promoter
  misc_feature 639..642: note = TATA
  misc_feature 759..1731: note = chicken beta-actin intron
  misc_feature 1749..2084: note = cIns(N2-1)
  misc_feature 2136..2262: note = rabbit globin polyA
  source 1..2262: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctcgggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatgagacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 31 - DNA, 2677 bp, synthetic construct
  misc_feature 1..2677: note = ITR.CB7.CI.cINS2-1(N).RBG .ITR
  misc_feature 1..130: note = AAV2 - shortened 5' ITR
  misc_feature 198..579: note = CMV IE promoer
  misc_feature 198..863: note = CB7 promoter element
  misc_feature 582..863: note = CB promoter
  misc_feature 836..839: note = TATA
  misc_feature 956..1928: note = chicken beta actin intron
  misc_feature 1946..2281: note = canine preinsulin
  misc_feature 2333..2459: note = rabbit globin polyA
  misc_feature 2548..2677: note = AAV2 - 3' ITR
  misc_feature 2548..2677: note = AAV2 - shortened 3' ITR
  source 1..2677: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctcgggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaactgatgagacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - protein, 708 aa, synthetic construct
  REGION 1..708: note = feline insulin with leader and fused to feline serum albumin
  SIGNAL 1..24
  REGION 25..110: note = MISC_FEATURE - modified feline insulin
  REGION 111..124: note = MISC_FEATURE - modified feline insulin
  REGION 125..708: note = MISC_FEATURE - feline serum albumin
MAPWTRLLPLLALLSLWIPAPTRAFVNQHLCGSHLVEALYLVCGERGFFYTPRAKREAEDLQGKDAELGEAPGAGGLQPSALEAPRQKRGIVEQCCASVCSLYQLEHYCNGGGGSGGGGSGGGSEAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 33 - DNA, 2127 bp, synthetic construct
  misc_feature 1..2127: note = feline mutant insulin - albumin fusion protein coding sequence
  source 1..2127: mol_type = other DNA
atggctccttggacgaggctgctaccgctgttggctcttctgtccctctggatacccgctcccacccgtgcatttgtcaatcagcacctctgtgggtcccaccttgttgaggccctctatctggtctgcggggagagagggttcttctacacgccccgggctaagcgcgaggcagaggacttgcagggaaaggacgctgagctcggggaagcccccggggcgggcggcctacaaccctccgccctggaggctcctcggcaaaagcgcggaattgtagagcagtgttgtgcctccgtgtgctcactctaccagctcgaacactattgtaacggggggggagggtcggggggcggcgggagcggaggcggcagcgaagctcaccagagcgaaatcgcccaccggtttaacgatttaggtgaggagcacttcagggggctggttttggttgccttcagccagtatctgcagcaatgtccattcgaggatcacgtgaagttggtgaatgaggtcactgagttcgccaaaggctgtgttgccgatcaatcagctgccaactgcgagaaatctctgcacgaacttctcggcgacaagctgtgcacggtggcctccctgcgagacaaatacggcgaaatggccgactgctgtgaaaagaaagaaccagaacgcaacgagtgtttcctccagcacaaggacgacaaccctggctttgggcaattagttaccccggaggccgacgctatgtgcaccgcattccacgaaaatgagcaacggtttctggggaagtacctctatgagatcgcacgtcgtcacccatatttttatgccccagaattactgtactatgccgaggaatataagggggtgttcaccgaatgctgcgaagccgcggacaaggctgcctgtcttactcctaaagtggacgcattacgggaaaaagtacttgctagctcagctaaagagagactgaagtgtgcttcactgcagaagtttggagaacgagctttcaaagcctggtccgtggctcggctatcgcagaaatttcccaaggctgagttcgcggagatctcaaaactggtcacagaccttgctaaaatacataaagaatgctgccacggcgacctgcttgagtgtgctgatgatcgcgccgacctggcgaaatacatatgcgagaatcaggactctatctcaacaaaactgaaagagtgttgtggtaaaccagtgttggagaaaagccattgcatttcagaggtggagcgcgacgaacttccagcggacctgccgcccttggctgtagacttcgttgaggataaggaagtgtgtaaaaactatcaggaagctaaagacgtgtttctgggcacctttctttacgagtattctaggaggcatcctgagtattctgtgagtcttctgctcaggctcgcgaaggagtacgaagcaacacttgagaaatgctgcgccacggatgatcctcccgcctgttatgcacacgtgttcgatgagttcaagcccctggttgaagagcctcacaacttggtgaagaccaattgcgaactgtttgagaaactgggggagtacggctttcagaatgctttactggtgcgctatactaagaaagttccccaagtctctacacccactctggtagaggtgagcaggtcgctgggaaaggtgggctcaaaatgctgtacccatcctgaggctgagcgtctgagttgtgccgaggattacttatccgtcgtgctgaatagactgtgcgtcctccacgagaaaaccccggtgagcgaaagggtaaccaagtgttgtaccgaatcactggttaataggaggccttgcttctccgctttgcaggtggacgaaacctatgtgcctaaggagttctccgccgaaacatttaccttccacgccgacctctgcactttacccgaagcagagaagcagataaagaagcagtccgctctagtcgaattgctgaaacataagccaaaagcaacagaagagcagcttaaaaccgtgatgggcgacttcggttccttcgttgacaagtgctgtgccgccgaggacaaagaagcgtgcttcgccgaggaaggtcctaagctggtcgcggcggctcaggcggcattggcttga

SEQ ID NO: 34 - protein, 584 aa, Felis catus
EAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 35 - DNA, 2211 bp, Adeno-Associated Virus hu68
  source 1..2211: mol_type = unassigned DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 36 - DNA, 2211 bp, Adeno-Associated Virus hu68
  source 1..2211: mol_type = unassigned DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 37 - protein, 736 aa, Adeno-Associated Virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2025/0388926 A1
(43) Pub. Date: Dec. 25, 2025

(54) VIRAL VECTOR GENOME ENCODING AN INSULIN FUSION PROTEIN

(71) Applicants: The Trustees of the University of Pennsylvania, Philadelphia, PA (US); Scout Bio, Inc., Philadelphia, PA (US)

(72) Inventors: Christian Hinderer, Baltimore, MD (US); Makoto Horiuchi, Wallingford, PA (US); Samantha Busfield, Philadelphia, PA (US); Matthew J. Wilson, Philadelphia, PA (US)

(21) Appl. No.: 18/843,040

(22) PCT Filed: Mar. 1, 2023
(86) PCT No.: PCT/US2023/063516

§ 371 Date: Aug. 30, 2024

Related U.S. Application Data
(60) Provisional application No. 63/315,252, filed on Mar. 1, 2022.

(51) Int. Cl. C12N 15/86; A61K 38/28; A61K 38/38; A61K 48/00; A61P 3/10; C07K 14/005; C07K 14/62; C07K 14/765

(52) U.S. Cl. CPC C12N 15/86; A61K 38/28; A61K 38/385; A61K 48/005; A61P 3/10; C07K 14/005; C07K 14/62; C07K 14/765; C07K 2319/02; C12N 2750/14122; C12N 2750/14143

(57) ABSTRACT

Provided are compositions and methods for treating diabetes in a canine or feline. A viral particle comprising a polynucleotide encoding a canine or feline insulin-serum albumin fusion polypeptide is administered to a subject.

### REFERENCE TO AN ELECTRONIC SEQUENCE LISTING

The contents of the electronic sequence listing (UPN-22-10022.PCT.xml; Size: 111 kb; and Date of Creation: Mar. 1, 2023) is herein incorporated by reference in its entirety.

### FIELD OF INVENTION

The invention relates generally to compositions and methods for treating diabetes in a canine or feline.

### BACKGROUND

Diabetes mellitus is a syndrome associated with protracted hyperglycemia due to loss or dysfunction of insulin secretion by pancreatic beta cells, diminished insulin sensitivity in tissues, or both. In the dog, beta-cell loss tends to be rapid and progressive, and is usually due to immune-mediated destruction, vacuolar degeneration, or pancreatitis. In the cat, loss or dysfunction of beta cells is the result of insulin resistance, islet amyloidosis, or chronic lymphoplasmacytic pancreatitis.

Insulin is an endogenous peptide hormone produced by beta cells of the pancreatic islets and it is considered to be the main anabolic hormone of the body. Insulin is the mainstay of therapy for diabetic dogs and cats. The current standard of care is twice daily insulin injections by the dog or cat's caregiver along with frequent veterinarian visits and disposable diagnostics that are expensive, time consuming and inconvenient.

The present disclosure provides compositions and methods related to a virion engineered to provide sustained expression of insulin.

### SUMMARY OF THE INVENTION

In certain embodiments, a recombinant adeno-associated viral (rAAV) virion is provided for treatment of companion animals. The rAAV comprises an AAV capsid and a vector genome comprising an expression cassette comprising a polynucleotide encoding a fusion protein comprising a proinsulin and serum albumin, the expression cassette flanked by a 5′ inverted terminal repeat (ITR) and a 3′ ITR, wherein the proinsulin is a canine proinsulin or a feline proinsulin. In certain embodiments, the AAV capsid is selected for its ability to express in muscle cells. In certain embodiments, the capsid is a AAVrh91 capsid.

In certain embodiments, the proinsulin is canine proinsulin. In other embodiments, the proinsulin is feline proinsulin. Suitably, the canine or feline proinsulin comprises an N-terminal signal peptide. In certain embodiments, the N-terminal signal peptide for canine insulin is a canine insulin signal peptide. In certain embodiments, the N-terminal signal peptide for feline insulin is a feline signal peptide.

In certain embodiments, the canine proinsulin is a canine proinsulin variant having a mutation at one or more cleavage sites compared to a reference polypeptide sequence as set forth in SEQ ID NO: 10. In certain embodiments, the proinsulin is a canine proinsulin fused to a canine serum albumin. In certain embodiments, the proinsulin-serum albumin fusion polynucleotide shares at least 80%, at least 85%, at least 90%, at least 95%, at least 99%, or 100% identity to SEQ ID NO: 2. In certain embodiments, the proinsulin comprises K29R, R31K, and L62R mutations compared to a reference polypeptide sequence as set forth in SEQ ID NO: 10. In certain embodiments, the canine proinsulin-serum albumin fusion protein comprises a linker that shares at least 80%, at least 85%, at least 90%, at least 95%, at least 99%, or 100% identity to SEQ ID NO: 8.

In certain embodiments, the feline proinsulin is a variant having a mutation at one or more cleavage sites compared to a reference polypeptide sequence as set forth in SEQ ID NO: 24. In certain embodiments, the proinsulin is a feline proinsulin fused to a feline serum albumin.

In certain embodiments, the feline proinsulin-feline serum albumin fusion polynucleotide shares at least 80%, at least 85%, at least 90%, at least 95%, at least 99%, or 100% identity to SEQ ID NO: 33. The feline proinsulin may comprise K29R, R31K, and L62R mutations compared to a reference polypeptide sequence as set forth in SEQ ID NO: 24.

In certain embodiments, the feline proinsulin-feline serum albumin fusion polynucleotide shares at least 80%, at least 85%, at least 90%, at least 95%, at least 99%, or 100% identity to SEQ ID NO: 33. The feline proinsulin may comprises K29R, R31K, and L62R mutations compared to a reference polypeptide sequence as set forth in SEQ ID NO: 24. The polynucleotide encoding the fusion protein is operatively linked to a promoter. In certain embodiments, the promoter is a CB7 promoter element comprising a cytomegalovirus enhancer/and a chicken b-actin promoter. In certain embodiments, the expression cassette comprises polynucleotide sequence encoding a homology directed repair (HDR) template configured for insertion into a cut site.

In certain embodiments, a pharmaceutical composition suitable for use in treating a metabolic disease in a canine or feline is provided which comprises the rAAV virion. The rAAV virion or the pharmaceutical composition may be used in a method for treating a canine or feline subject having a metabolic disease, optionally diabetes.

Certain embodiments provide for use of the rAAV virion or the pharmaceutical composition in the manufacture of a medicament for treating a canine or feline subject having a metabolic disease, optionally diabetes. Certain embodiments provide for the rAAV virion to be in a composition formulated to be administered to the canine or feline subject at a dose of 1×10⁹ GC/kg to 3×10¹³ GC/kg of the rAAV and/or wherein the rAAV is delivered intramuscularly.

In certain embodiments, a method of treating a canine or feline subject having a metabolic disease is provided which comprises administering to the canine or feline subject an effective amount of the rAAV virion or the pharmaceutical composition. In certain embodiments, the method is for treatment of a metabolic disease, e.g., diabetes. In certain embodiments, the diabetes is Type 1 diabetes. In other embodiments, the diabetes is Type 2 diabetes. In certain embodiments, the effective amount is administered intramuscularly. In certain embodiments, the effective amount is between 1×10⁹ GC/kg to 3×10¹³ GC/kg of the rAAV virion. In certain embodiments, the effective amount is between 1×10¹⁰ GC/kg to 3×10¹³ GC/kg of the rAAV virion. In certain embodiments, the method results in expression of the fusion protein in the subject for at least one week, at least two weeks, at least four weeks, at least 6 weeks, at least 8 weeks, at least 10 weeks, at least 12 weeks, at least 16 weeks, at least 20 weeks, at least 30 weeks, at least 40 weeks, at least 50 weeks, or at least 60 weeks. In certain embodiments, the method results in expression of the fusion protein in the subject at a therapeutically effective concentration for at least three months, at least six months, or at least twelve months. In certain embodiments, the method decreases fasting blood glucose in the subject by about 10%, about 15%, about 20%, about 25%, about 30%, about 35%, about 40%, about 45%, or about 50%.

Still further aspects and advantages of the invention will be apparent from the following detailed disclosure of the invention.

### BRIEF DESCRIPTION OF THE DRAWINGS

FIGS. 1A-1C show schematic diagrams of illustrative canine insulin proteins of the present disclosure. All three proteins incorporate a native signal peptide (SP) and modified furin sites. FIG. 1A shows a schematic diagram of an illustrative canine preproinsulin-serum albumin fusion protein (cINS-Alb). The caINS-Alb protein has a glycine/serine linker that links the A-chain of insulin to canine serum albumin. FIG. 1B shows a schematic diagram of an illustrative canine preproinsulin-transferrin fusion protein (cINS-Tf). The caINS-Tf protein has a glycine/serine linker that links the A-chain of insulin to canine transferrin. FIG. 1C shows a schematic diagram of an illustrative canine preproinsulin protein containing the furin site modifications and serves as a control (cINS-2-1).

FIG. 2 shows in vitro insulin bioactivity of caINS-Alb and caINS-Tf compared to a control insulin standard. EC₅₀ values are listed in the table below the graph. Ligand-induced activation of the insulin receptor in response to increasing concentrations of purified cINS-Alb and cINS-Tf. A reference insulin standard was used as a control. Relative potency is represented as Relative Light Units.

FIG. 3A shows blood glucose levels of Streptozotocin (STZ) NOD-SCID mice administered AAV virions containing canine insulin analogs or PBS control. In vivo activity of AAV-cINS analogs (cINS-Alb and cINS-Tf) in NOD-SCID Diabetic Mice was observed. Following intramuscular (IM) administration of AAV candidates at Day 0, fasting blood glucose was monitored for the duration of the study. Data shown is mean and standard deviation (SD) of cohorts (n=7-10/group).

FIG. 3B shows body weight of STZ NOD-SCID mice administered AAV virions containing canine insulin analogs or PBS control. In vivo activity of AAV-cINS analogs (cINS-Alb and cINS-Tf) in NOD-SCID Diabetic Mice was observed. Following intramuscular (IM) administration of AAV candidates at Day 0, body weight was monitored for the duration of the study. Data shown is mean and standard deviation (SD) of cohorts (n=7-10/group).

FIGS. 3C and 3D show ex vivo serum insulin activity of STZ NOD-SCID mice administered AAV virions containing canine insulin analogs or PBS control. In vivo activity of AAV-cINS analogs (cINS-Alb and cINS-Tf) in NOD-SCID Diabetic Mice was observed. Following intramuscular (IM) administration of AAV candidates at Day 0, Serum samples were taken at Day 28 (FIG. 3C) and Day 56 (FIG. 3D) and were assayed for insulin bioactivity. Relative insulin activity is expressed as Relative Light Units. Data shown is mean and spread of individual mice. Statistical difference is shown between the control cINS-2-1 and cINS-Alb at both timepoints (analyzed with one-way ANOVA).

### DETAILED DESCRIPTION OF THE INVENTION

As described elsewhere herein, the present disclosure is predicated, at least in part, on the inventors' surprising finding that virions encoding insulin fusion proteins achieve sustained expression of insulin in canines and felines. Provided are methods of making and using such virions.

An insulin fusion protein engineered to overcome the short half-life of the native hormone by fusion to a protein with longer half-life (e.g., serum albumin) is a therapeutic advancement for the treatment of diabetes. Long-acting insulin fusion protein expression constructs have been developed for use in canine and feline animals. The expression constructs comprise a secretion signal peptide, as well as a fusion domain intended to prolong the time in circulation of the resulting fusion protein.

The expression constructs are delivered to subjects in need thereof via transduction of a viral particle, such as an AAV particle, and in vivo expression of the encoded insulin fusion protein. Also provided are methods of using these constructs in regimens for treating type 1 diabetes mellitus (T1DM), type 2 diabetes mellitus (T2DM), or metabolic syndrome in a veterinary subject and increasing the half-life of insulin in a subject.

The present invention encompasses insulin-albumin fusion proteins comprising a therapeutic protein having insulin activity. The present invention also encompasses polynucleotides comprising, or alternatively consisting of, nucleic acid molecules encoding a therapeutic protein having insulin activity fused to albumin or a fragment (portion) or variant of albumin. Albumin may be fused to the N-terminus, the C-terminus, or both termini of the therapeutic protein having insulin activity. In some embodiments, the albumin is fused to the C-terminus of the proinsulin. The present invention also encompasses polynucleotides, comprising nucleic acid molecules encoding proteins comprising a therapeutic protein having insulin activity fused to albumin or a fragment (portion) or variant of albumin, that is sufficient to prolong its activity in vivo.

### Leader Sequence

In one embodiment, the insulin protein comprises a leader sequence, which may comprise a secretion signal peptide. As used herein, the term “leader sequence” refers to any N-terminal sequence of a polypeptide. In one embodiment, the canine or feline insulin proteins described herein comprise a leader, or signal sequence, and proinsulin. The leader sequence is, in one embodiment, a native sequence (canine or feline insulin) leader. In another embodiment, the leader sequence is a heterologous sequence, i.e., derived from another protein than canine or feline insulin.

In one embodiment, the leader is a canine IL-2 sequence. In one embodiment, the IL-2 leader comprises a sequence that shares at least 80%, at least 85%, at least 90%, at least 95%, at least 99%, or 100% identity to SEQ ID NO: 12.

SEQ ID NO: 12:
MYKMQLLSCIALTLVLVANS

In another embodiment, the leader is the native canine insulin sequence. In one embodiment, the canine leader comprises a sequence that shares at least 80%, at least 85%, at least 90%, at least 95%, at least 99%, or 100% identity to SEQ ID NO: 7.

SEQ ID NO: 7:
MALWMRLLPLLALLALWAPAPTRA

In one embodiment, the leader sequence is a feline IL-2 sequence. In one embodiment, the IL-2 leader comprises a sequence that shares at least 80%, at least 85%, at least 90%, at least 95%, at least 99%, or 100% identity to SEQ ID NO: 13.

SEQ ID NO: 13:
MYKIQLLSCIALTLILVINS

In another embodiment, the leader is the native feline insulin sequence. In one embodiment, the canine leader comprises a sequence that shares at least 80%, at least 85%, at least 90%, at least 95%, at least 99%, or 100% identity to SEQ ID NO: 9.

SEQ ID NO: 9:
MAPWTRLLPLLALLSLWIPAPTRA

The leader sequence may be derived from the same species for which administration is ultimately intended, i.e., a canine or feline animal. As used herein, the terms “derived” or “derived from” mean the sequence or protein is sourced from a specific subject species or shares the same sequence as a protein or sequence sourced from a specific subject species. For example, a leader sequence which is “derived from” a canine or feline, shares the same sequence (or a variant thereof, as defined herein) as the same leader sequence as expressed in a canine or feline. However, the specified nucleic acid or amino acid need not actually be sourced from a canine or feline. Various techniques are known in the art which are able to produce a desired sequence, including mutagenesis of a similar protein (e.g., a homolog) or artificial production of a nucleic acid or amino acid sequence. The “derived” nucleic acid or amino acid retains the function of the same nucleic acid or amino acid in the species from which it is “derived”, regardless of actual source of the derived sequence.

### Insulin

Insulin is involved in regulation of glucose utilization in the body. The body's inability to synthesize insulin or cells that are resistant to insulin leads to diabetes mellitus which is characterized by chronic hyperglycemia. Preproinsulin is transcribed as a 110 amino acid chain having an N-terminal signal peptide. Removal of the signal peptide from its N-terminus produces proinsulin. Formation of disulfide bonds between the A-& B-chain components, and removal of the intervening C-chain, produces a biologically active insulin molecule comprising 51 amino acids, in size less than half of the original translation product.

Unless otherwise specified, the term “insulin” refers to a biologically active insulin molecule or a functional fragment thereof which provides a desired insulin-like activity, and amino-acid sequence variants of these molecules. The disclosure provides proteins comprising canine insulin or feline insulin, as well as polynucleotides and expression vectors encoding such proteins. In some embodiments, the insulin protein comprises a polynucleotide sequence encoding a polypeptide comprising (a) a secretion signal peptide, (b) a proinsulin polypeptide, (c) an optional linker, and (d) an optional fusion partner.

In one embodiment, the protein comprises a canine IL2 signal peptide and canine proinsulin. In another embodiment, the protein comprises a canine insulin signal peptide and canine proinsulin. The amino acid sequence of native canine proinsulin is shown in SEQ ID NO: 10. In another embodiment, the protein comprises a feline IL2 signal peptide and feline proinsulin. In another embodiment, the protein comprises a feline insulin signal peptide and feline proinsulin. The amino acid sequence of native feline proinsulin is shown in SEQ ID NO: 24.

In some embodiments, canine or feline insulin includes variants which may include up to about 10% variation from an insulin nucleic acid or amino acid sequence described herein or known in the art, which retain the function of the wild-type sequence. As used herein, by “retain function” it is meant that the nucleic acid or amino acid functions in the same way as the wild type sequence, although not necessarily at the same level of expression or activity. For example, in one embodiment, a functional variant has increased expression or activity as compared to the wild type sequence. In another embodiment, the functional variant has decreased expression or activity as compared to the wild type sequence. In one embodiment, the functional variant has 10%, 20%, 30%, 40%, 50%, 60%, 70%, 80%, 90% or greater increase or decrease in expression or activity as compared to the wild type sequence [SEQ ID NO: 24].

The canine proinsulin sequence, in one embodiment, contains one or more mutations as compared to the native sequence. These mutations are, in some embodiments, in the cleavage sites between the B/C chains and C/A chains. In one embodiment, one or more of the cleavage sites are mutated to incorporate at least one furin cleavage site at existing protease cleavage sites. In one embodiment, the proinsulin sequence has a K29R mutation. In another embodiment, the proinsulin sequence has a R31K mutation. In another embodiment, the proinsulin sequence has a L62R mutation. In another embodiment, the proinsulin sequence has both K29R and R31K mutations. In another embodiment, the proinsulin sequence has both K29R and L62R mutations. In another embodiment, the proinsulin sequence has both R31K and L62R mutations. In another embodiment, the proinsulin sequence has K29R, R31K, and L62R mutations.

In one embodiment, the canine proinsulin sequence is a sequence that shares at least 80%, at least 85%, at least 90%, at least 95%, at least 99%, or 100% identity SEQ ID NO: 1, 3, 10 or 14.

In one embodiment, the feline proinsulin sequence is a sequence that shares at least 80%, at least 85%, at least 90%, at least 95%, at least 99%, or 100% identity SEQ ID NO: 15, 25, or 33.

When a variant or fragment of the proinsulin sequence is desired, the coding sequences for these peptides may be generated using site-directed mutagenesis of the wild-type nucleic acid sequence. Alternatively, or additionally, web-based or commercially available computer programs, as well as service-based companies may be used to back translate the amino acids sequences to nucleic acid coding sequences, including both RNA and/or cDNA. See, e.g., backtranseq by EMBOSS; Gene Infinity; and/or ExPasy. In one embodiment, the RNA and/or cDNA coding sequences are designed for optimal expression in the subject species for which administration is ultimately intended, i.e., a canine or a feline.

### Fusion Domains

The disclosure provides fusion proteins comprising a fusion domain. By fusing an insulin to a fusion domain with longer half-life, the insulin fusion protein overcomes the short half-life of the native hormone. In some embodiments, the fusion domain comprises either (i) a canine serum albumin or a functional variant thereof, (ii) a canine IgG Fc or a functional variant thereof, or (iii) a canine transferrin or a functional variant thereof. In some embodiments, the fusion domain comprises a canine serum albumin.

In some embodiments, the fusion domain comprises either (i) a feline serum albumin or a functional variant thereof, (il) a feline IgG Fc or a functional variant thereof, or (iii) a feline transferrin or a functional variant thereof. In some embodiments, the fusion domain comprises a feline serum albumin.

In some embodiments, the fusion domain is a canine serum albumin comprising a sequence that shares at least 80%, at least 85%, at least 90%, at least 95%, at least 99%, or 100% identity to SEQ ID NO: 16.

In some embodiments, the fusion domain is a canine transferrin comprising a sequence that shares at least 80%, at least 85%, at least 90%, at least 95%, at least 99%, or 100% identity to SEQ ID NO: 17.

In some embodiments, the fusion domain is a feline serum albumin comprising a sequence that shares at least 80%, at least 85%, at least 90%, at least 95%, at least 99%, or 100% identity to SEQ ID NO: 18.

### Insulin Fusion Proteins

The disclosure provides fusion proteins comprising one or more copies of proinsulin, as well as polynucleotides and expression vectors encoding such fusion proteins. In some embodiments, the fusion protein comprises a polynucleotide sequence encoding a fusion protein comprising (a) a leader sequence comprising a secretion signal peptide, (b) a proinsulin, and (c) a fusion domain comprising either (i) an IgG Fc or a functional variant thereof, (ii) an albumin or a functional variant thereof, or (iii) a transferrin or a functional variant thereof. In one embodiment, the fusion protein comprises a thrombin leader sequence, a proinsulin, and an IgG Fc or functional variant thereof. In another embodiment, the fusion protein comprises a thrombin leader sequence, a proinsulin, and an albumin or functional variant thereof.

In some embodiments, the fusion protein comprises a canine insulin leader sequence, a canine proinsulin (K29R, R31K and L62R, with respect to the numbering of SEQ ID NO: 10), a glycine/serine linker, and a canine serum albumin. In an embodiment, the fusion protein comprises a sequence that shares at least 80%, at least 85%, at least 90%, at least 95%, at least 99%, or 100% identity to SEQ ID NO: 1.

In some embodiments, the fusion protein comprises a canine insulin leader sequence, a canine proinsulin (K29R, R31K and L62R, with respect to the numbering of SEQ ID NO: 10), a glycine/serine linker, and a canine transferrin. In an embodiment, the fusion protein comprises a sequence that shares at least 80%, at least 85%, at least 90%, at least 95%, at least 99%, or 100% identity to SEQ ID NO: 3.

In some embodiments, the fusion protein comprises a canine insulin leader sequence and a canine proinsulin (K29R, R31K and L62R, with respect to the numbering of SEQ ID NO: 10). In an embodiment, the fusion protein comprises a sequence that shares at least 80%, at least 85%, at least 90%, at least 95%, at least 99%, or 100% identity to SEQ ID NO: 5.

In some embodiments, the fusion protein comprises a feline insulin leader sequence, a feline proinsulin (K29R, R31K and L62R, with respect to the numbering of SEQ ID NO: 24), a glycine/serine linker, and a feline serum albumin. In an embodiment, the fusion protein comprises a sequence that shares at least 80%, at least 85%, at least 90%, at least 95%, at least 99%, or 100% identity to SEQ ID NO: 24 or 25.

In one embodiment, the fusion protein comprises an insulin leader sequence, a proinsulin, and an albumin or functional variant thereof. In one embodiment, the fusion protein comprises an insulin leader sequence, a proinsulin, and a transferrin or functional variant thereof.

In one embodiment, the fusion protein comprises an IL2 leader sequence, a proinsulin, and an albumin or functional variant thereof. In one embodiment, the fusion protein comprises an IL2 leader sequence, a proinsulin, and a transferrin or functional variant thereof.

In addition to the leader sequence, proinsulin, and insulin polypeptides provided herein, nucleic acid sequences (used interchangeably with “polynucleotides”) encoding these polypeptides are provided. In one embodiment, a nucleic acid sequence is provided which encodes for the proinsulin-serum albumin fusion polypeptide described herein. In some embodiments, the nucleic acid sequence which encodes the canine proinsulin-serum albumin fusion comprises a sequence that shares at least 80%, at least 85%, at least 90%, at least 95%, at least 99%, or 100% identity to SEQ ID NO: 2.

In some embodiments, the nucleic acid sequence which encodes the canine proinsulin-transferrin fusion comprises a sequence that shares at least 80%, at least 85%, at least 90%, at least 95%, at least 99%, or 100% identity to SEQ ID NO: 4.

The in vivo function and stability of the fusion proteins of the present disclosure may be optimized by adding small peptide linkers, e.g., to prevent potentially unwanted domain interactions or for other reasons. Further, a glycine-rich linker may provide some structural flexibility such that the proinsulin portion can interact productively with the insulin receptor on target cells. Thus, the C-terminus of the proinsulin and the N-terminus of the fusion domain of the fusion protein are, in one embodiment, fused via a linker. In some embodiments, the linker includes 1, 2, 3 . . . or n repeats of a G-rich peptide linker having the sequence (GGGGS)n. In one embodiment, the linker includes 1, 1.5 or 2 repeats of a G-rich peptide linker having the sequence GGGGSGGGGSGGGGS (SEQ ID NO: 19). In one embodiment, the linker includes repeats of a G-rich peptide linker having the sequence GGGGGGGGSGGGS (SEQ ID NO 8).

### Vector Genome

The vector genome may comprise a monocistronic or polycistronic transcript. In some embodiments, the viral particles of the present disclosure comprise a polynucleotide sequence encoding, in 5′ to 3′:
(a) a CB7 promoter element comprising a cytomegalovirus (CMV) enhancer and a chicken b-actin promoter;
(b) canine insulin signal peptide;
(c) canine proinsulin (K29R, R31K and L62R);
(d) Gly/Ser linker; and
(e) canine serum albumin.

In certain embodiments, the recombinant AAV vectors (particles) comprise an AAV capsid and a vector genome comprising: a 5′ AAV ITR, a promoter, an enhancer, an optional intron, an open reading frame which is a nucleic acid sequence encoding a fusion protein comprising a canine insulin signal peptide, a canine proinsulin, a linker, and canine serum albumin, a polyA, and a 3′ AAV ITR. In certain embodiments, the canine proinsulin is the engineered canine proinsulin (K29R, R31K and L62R).

In certain embodiments, the recombinant AAV vectors (particles) comprise an AAV capsid and a vector genome comprising: a 5′ AAV ITR, a promoter, an enhancer, an optional intron, an open reading frame which is a nucleic acid sequence encoding a fusion protein comprising a feline insulin signal peptide, a feline proinsulin, a linker, and feline serum albumin, a polyA, and a 3′ AAV ITR. In certain embodiments, the feline proinsulin is the engineered feline proinsulin (K29R, R31K and L62R).

In some embodiments, the viral particle comprises a nucleic acid sequence that shares at least 75%, at least 80%, at least 85%, at least 90%, at least 95%, at least 99%, or 100% identity to SEQ ID NO: 26 (CB7.CI.cINS2-1_3×GSsp.ALB.rBG) or 28 (CB7.CI.cINS2-1_3×GSsp.Tf.rBG).

In certain embodiments, the vector genome of the production plasmid includes a shortened 5′ and/or 3′ AAV ITR of 130 base pairs, wherein the external A elements is/are deleted. In certain embodiments, the shortened ITR reverts back to the wild type length of 145 base pairs during vector DNA amplification using the internal A element as a template. Thus, the final viral particles generated from a production plasmid comprising the shorted ITRs contains the full-length 145 ITRs (by adding 15 nucleotides to the extreme 3′ and/or 15 nucleotides to the extreme 5′ end of the vector genome). In other embodiments, the full-length AAV 5′ and 3′ ITRs are used. The ITRs are selected such that they are transcomplemented by the rep protein expressed during production (e.g., in a trans plasmid). Where the source of the ITRs is from AAV2 and the AAV capsid is from another AAV source, the resulting rAAV may be termed pseudotyped. However, other configurations of these elements may be suitable.

### Adeno-Associated Virus (AAV)

In one aspect, virions comprising polynucleotides which encode the leader sequences, proinsulin, and insulin polypeptides as described herein, are provided. In certain embodiments of the virions described herein, the virion is an adeno-associated virus (AAV) or a recombinant AAV (rAAV). The term “recombinant AAV” or “rAAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) virion is an AAV DNase-resistant viral particle having an AAV protein capsid into which is packaged an expression cassette flanked by AAV inverted terminal repeat sequences (ITRs) (together referred to as the “vector genome”) for delivery to target cells.

An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral particles as identified above. In one embodiment, the AAV capsid is an AAVrh91 capsid or variant thereof. See, e,g, WO2020/223232 (for AAVrh91 predicted amino acid sequence and deamidation pattern of assembled AAVrh91 capsid) and WO2022/036220. In certain embodiments, the capsid protein is designated by a number or a combination of numbers and letters following the term “AAV” in the name of the rAAV virion. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAVAnc80, AAV10, AAV11, AAV12, AAVrh8, AAVrh74, AAV-DJ8, AAV-DJ, AAVhu37, AAVrh64R1, and AAVhu68.

Illustrative AAV virions are described in e.g., US Published Patent Application No. 2007-0036760-A1, US Published Patent Application No. 2009-0197338-A1, EP 1310571, WO 2003/042397 (AAV7 and other simian AAV), U.S. Pat. Nos. 7,790,449, 7,282,199 (AAV8), WO 2005/033321, U.S. Pat. No. 7,906,111 (AAV9), WO 2006/110689, WO 2003/042397 (rh10), WO 2005/033321, WO 2018/160582 (AAVhu68), which are incorporated herein by reference.

Other suitable AAVs may include, without limitation, AAVrh90, AAVrh91, AAVrh92, AAVrh93, AAVrh91.93. Other suitable AAVs include AAV3B variants which are described in International Patent Application No. PCT/US20/56511, filed Oct. 20, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. These documents also describe other AAV capsids which may be selected for generating rAAV and are incorporated by reference.

As used herein, relating to AAV, the term “variant” means any AAV sequence which is derived from a known AAV sequence, including those with a conservative amino acid replacement, and those sharing at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

In one embodiment, the virion is an rAAV having the capsid of AAVrh91 or a functional variant thereof. In one embodiment, the virion is an rAAV having the capsid of AAV3.AR.2.12 or a functional variant thereof. In one embodiment, the virion is an rAAV having the capsid of AAV8 or a functional variant thereof. In one embodiment, the virion is an rAAV having a capsid selected from AAV9, AAVrh64R1, AAVhu37, or AAVrh10. In one embodiment, the virion is an rAAV having the capsid of AAVhu68 or a functional variant thereof.

In certain embodiments, the viral vector has an AAVrh91 capsid. A nucleic acid sequence encoding the AAVrh91 capsid is provided in SEQ ID NO: 21 and the encoded amino acid sequence is provided in SEQ ID NO: 22. Provided herein is an rAAV comprising at least one of the vp1, vp2 and the vp3 of AAVrh91 (SEQ ID NO: 22). Also provided herein are rAAV comprising an AAV capsid encoded by at least one of the vp1, vp2 and the vp3 of AAVrh91 (SEQ ID NO: 21). In yet another embodiment, a nucleic acid sequence encoding the AAVrh91 amino acid sequence is provided in SEQ ID NO: 22 and the encoded amino acid sequence is provided in SEQ ID NO: 22. Also provided herein are rAAV comprising an AAV capsid encoded by at least one of the vp1, vp2 and the vp3 of AAVrh91eng (SEQ ID NO: 23). In certain embodiments, the vp1, vp2 and/or vp3 is the full-length capsid protein of AAVrh91 (SEQ ID NO: 22). In other embodiments, the vp1, vp2 and/or vp3 has an N-terminal and/or a C-terminal truncation (e.g., truncation(s) of about 1 to about 10 amino acids)

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 22, vp1 proteins produced from SEQ ID NO: 21, or vp1 proteins produced from a nucleic acid sequence at least 70% identical to SEQ ID NO: 21 which encodes the predicted amino acid sequence of 1 to 736 of SEQ ID NO: 22, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 22, vp2 proteins produced from a sequence comprising at least nucleotides 412 to 2208 of SEQ ID NO: 21, or vp2 proteins produced from a nucleic acid sequence at least 70% identical to at least nucleotides 412 to 2208 of SEQ ID NO: 21 which encodes the predicted amino acid sequence of at least about amino acids 138 to 736 of SEQ ID NO: 22, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 22, vp3 proteins produced from a sequence comprising at least nucleotides 607 to 2208 of SEQ ID NO: 21, or vp3 proteins produced from a nucleic acid sequence at least 70% identical to at least nucleotides 607 to 2208 of SEQ ID NO: 21 which encodes the predicted amino acid sequence of at least about amino acids 203 to 736 of SEQ ID NO: 22; and/or (2) a heterogeneous population of vp1 proteins which are the product of a nucleic acid sequence encoding the amino acid sequence of SEQ ID NO: 22, a heterogeneous population of vp2 proteins which are the product of a nucleic acid sequence encoding the amino acid sequence of at least about amino acids 138 to 736 of SEQ ID NO: 22, and a heterogeneous population of vp3 proteins which are the product of a nucleic acid sequence encoding at least amino acids 203 to 736 of SEQ ID NO: 22, wherein: the vp1, vp2 and vp3 proteins contain subpopulations with amino acid modifications comprising at least two highly deamidated asparagines (N) in asparagine-glycine pairs in SEQ ID NO: 22 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 22, vp1 proteins produced from SEQ ID NO: 21, or vp1 proteins produced from a nucleic acid sequence at least 70% identical to SEQ ID NO: 21 which encodes the predicted amino acid sequence of 1 to 736 of SEQ ID NO: 22, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 22, vp2 proteins produced from a sequence comprising at least nucleotides 412 to 2208 of SEQ ID NO: 21, or vp2 proteins produced from a nucleic acid sequence at least 70% identical to at least nucleotides 412 to 2208 of SEQ ID NO: 21 which encodes the predicted amino acid sequence of at least about amino acids 138 to 736 of SEQ ID NO: 22, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 22, vp3 proteins produced from a sequence comprising at least nucleotides 607 to 2208 of SEQ ID NO: 21, or vp3 proteins produced from a nucleic acid sequence at least 70% identical to at least nucleotides 607 to 2208 of SEQ ID NO: 21 which encodes the predicted amino acid sequence of at least about amino acids 203 to 736 of SEQ ID NO: 22; and/or (2) a heterogeneous population of vp1 proteins which are the product of a nucleic acid sequence encoding the amino acid sequence of SEQ ID NO: 22, a heterogeneous population of vp2 proteins which are the product of a nucleic acid sequence encoding the amino acid sequence of at least about amino acids 138 to 736 of SEQ ID NO: 22, and a heterogeneous population of vp3 proteins which are the product of a nucleic acid sequence encoding at least amino acids 203 to 736 of SEQ ID NO: 22, wherein: the vp1, vp2 and vp3 proteins contain subpopulations with amino acid modifications comprising at least two highly deamidated asparagines (N) in asparagine-glycine pairs in SEQ ID NO: 22 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the AAVrh91 vp1, vp2 and vp3 proteins contain subpopulations with amino acid modifications comprising at least two highly deamidated asparagines (N) in asparagine-glycine pairs in SEQ ID NO: 22 and optionally further comprising subpopulations comprising other deamidated amino acids, wherein the deamidation results in an amino acid change. High levels of deamidation at N-G pairs N57, N383 and/or N512 are observed, relative to the number of SEQ ID NO: 22. Deamidation has been observed in other residues. In certain embodiments, AAVrh91 may have other residues deamidated, e.g., typically at less than 10% and/or may have other modifications, including phosphorylation (e.g., where present, in the range of about 2 to about 30%, or about 2 to about 20%, or about 2 to about 10%) (e.g., at S149), or oxidation (e.g, at one or more of ˜W22, ˜M211, W247, M403, M435, M471, W478, W503, ˜M537, ˜M541, ˜M559, ˜M599, M635, and/or, W695). Optionally the W may oxidize to kynurenine.

TABLE A
AAVrh91 Deamidation

| AAVrh91 | |
|---|---|
| Deamidation based on VP1 | |
| numbering | % Deamidation |
| N57 + Deamidation | 65-90, 70-95, 80-95, 75-100, 80-100, or |
| | 90-100 |
| N94 + Deamidation | 2-15 or 2-5 |
| N303 + Deamidation | 2-15 or 5-10 |
| N383 + Deamidation | 65-90, 70-95, 80-95, 75-100, 80-100, or |
| | 90-100 |
| N497 + Deamidation | 2-15 or 5-10 |
| N512 + Deamidation | 65-90, 70-95, 80-95, 75-100, 80-100, or |
| | 90-100 |
| ~N691 + Deamidation | 2-15, 2-10, or 5-10 |

In certain embodiments, an AAVrh91 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh91 sequence provided herein. See, SEQ ID NO: 22.

In certain embodiments, an AAVrh91 capsid comprises: a heterogeneous population of vp1 proteins which are the product of a nucleic acid sequence encoding the amino acid sequence of SEQ ID NO: 22, a heterogeneous population of vp2 proteins which are the product of a nucleic acid sequence encoding the amino acid sequence of at least about amino acids 138 to 736 of SEQ ID NO: 22, and a heterogeneous population of vp3 proteins which are the product of a nucleic acid sequence encoding at least amino acids 203 to 736 of SEQ ID NO: 22.

In certain embodiments, the modified AAVrh91 nucleic acid sequence is be used to generate a mutant rAAV having a capsid with lower deamidation than the native AAVrh91 capsid. Such mutant rAAV may have reduced immunogenicity and/or increase stability on storage, particularly storage in suspension form.

In certain embodiments, an AAV68 capsid is further characterized by one or more of the following. AAV hu68 capsid proteins comprise: AAVhu68 vp1 proteins produced by expression from a nucleic acid sequence which encodes the predicted amino acid sequence of 1 to 736 of SEQ ID NO: 37, vp1 proteins produced from SEQ ID NO: 35 or 36, or vp1 proteins produced from a nucleic acid sequence at least 70% identical to SEQ ID NO: 35 or 36 which encodes the predicted amino acid sequence of 1 to 736 of SEQ ID NO: 37; AAVhu68 vp2 proteins produced by expression from a nucleic acid sequence which encodes the predicted amino acid sequence of at least about amino acids 138 to 736 of SEQ ID NO: 37, vp2 proteins produced from a sequence comprising at least nucleotides 412 to 2211 of SEQ ID NO: 35 or 36, or vp2 proteins produced from a nucleic acid sequence at least 70% identical to at least nucleotides 412 to 2211 of SEQ ID NO: 35 or 36 which encodes the predicted amino acid sequence of at least about amino acids 138 to 736 of SEQ ID NO: 37, and/or AAVhu68 vp3 proteins produced by expression from a nucleic acid sequence which encodes the predicted amino acid sequence of at least about amino acids 203 to 736 of SEQ ID NO: 37, vp3 proteins produced from a sequence comprising at least nucleotides 607 to 2211 of SEQ ID NO: 35 or 36, or vp3 proteins produced from a nucleic acid sequence at least 70% identical to at least nucleotides 607 to 2211 of SEQ ID NO: 35 or 36 which encodes the predicted amino acid sequence of at least about amino acids 203 to 736 of SEQ ID NO: 37.

Additionally or alternatively, an AAV capsid is provided which comprise a heterogenous population of vp1 proteins optionally comprising a valine at position 157, a heterogenous population of vp2 proteins optionally comprising a valine at position 157, and a heterogenous population of vp3 proteins, wherein at least a subpopulation of the vp1 and vp2 proteins comprise a valine at position 157 and optionally further comprising a glutamic acid at position 67 based on the numbering of the vp1 capsid of SEQ ID NO: 37. Additionally or alternatively, an AAVhu68 capsid is provided which comprises a heterogenous population of vp1 proteins which are the product of a nucleic acid sequence encoding the amino acid sequence of SEQ ID NO: 37, a heterogenous population of vp2 proteins which are the product of a nucleic acid sequence encoding the amino acid sequence of at least about amino acids 138 to 736 of SEQ ID NO: 37, and a heterogenous population of vp3 proteins which are the product of a nucleic acid sequence encoding at least amino acids 203 to 736 of SEQ ID NO: 37, wherein: the vp1, vp2 and vp3 proteins contain subpopulations with amino acid modifications

The AAVhu68 vp1, vp2 and vp3 proteins are typically expressed as alternative splice variants encoded by the same nucleic acid sequence which encodes the full-length vp1 amino acid sequence of SEQ ID NO: 37 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 37 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 35 or 36), or a sequence at least 70% to at least 99% (e.g., at least 85%, at least 90%, at least 95%, at least 97%, at least 98% or at least 99%) identical to SEQ ID NO: 35 or 36 which encodes aa 203 to 736 of SEQ ID NO: 37. Additionally, or alternatively, the vp1-encoding and/or the vp2-encoding sequence may be co-expressed with the nucleic acid sequence which encodes the AAVhu68 vp2 amino acid sequence of SEQ ID NO: 37 (about aa 138 to 736) without the vp1-unique region (about aa 1 to about 137), or a strand complementary thereto, the corresponding mRNA or tRNA (nt 412 to 2212 of SEQ ID NO: 35 or 36), or a sequence at least 70% to at least 99% (e.g., at least 85%, at least 90%, at least 95%, at least 97%, at least 98% or at least 99%) identical to SEQ ID NO: 35 or 36 which encodes about aa 138 to 736 of SEQ ID NO: 37.

As described herein, a rAAVhu68 has a rAAVhu68 capsid produced in a production system expressing capsids from an AAVhu68 nucleic acid which encodes the vp1 amino acid sequence of SEQ ID NO: 37, and optionally additional nucleic acid sequences, e.g., encoding a vp3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 37. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 35 or 36, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 37 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 35 or 36). In certain embodiments, also provided is a nucleic acid sequence which encodes the AAVhu68 vp2 amino acid sequence of SEQ ID NO: 37 (about aa 138 to 736) without the vp1-unique region (about aa 1 to about 137), or a strand complementary thereto, the corresponding mRNA or tRNA (nt 412 to 2211 of SEQ ID NO: 35 or 36).

However, other nucleic acid sequences which encode the amino acid sequence of SEQ ID NO: 37 may be selected for use in producing rAAVhu68 capsids. In certain embodiments, the nucleic acid sequence has the nucleic acid sequence of SEQ ID NO: 35 or 36 or a sequence at least 70% to 99% identical, at least 75%, at least 80%, at least 85%, at least 90%, at least 95%, at least 97%, at least 99%, identical to SEQ ID NO: 35 or 36 which encodes SEQ ID NO: 37. In certain embodiments, the nucleic acid sequence has the nucleic acid sequence of SEQ ID NO: 35 or 36 or a sequence at least 70% to 99. %, at least 75%, at least 80%, at least 85%, at least 90%, at least 95%, at least 97%, at least 99%, identical to about nt 412 to about nt 2211 of SEQ ID NO: 35 or 36 which encodes the vp2 capsid protein (about aa 138 to 736) of SEQ ID NO: 37. In certain embodiments, the nucleic acid sequence has the nucleic acid sequence of about nt 607 to about nt 2211 of SEQ ID NO: 35 or 36 or a sequence at least 70% to 99. %, at least 75%, at least 80%, at least 85%, at least 90%, at least 95%, at least 97%, at least 99%, identical to nt SEQ ID NO: 35 or 36 which encodes the vp3 capsid protein (about aa 203 to 736) of SEQ ID NO: 37.

| Deamidation | | |
|---|---|---|
| Based on Predicted | Average % Based on VP1/VP2/VP3 | |
| AAVHu68 [SEQ ID NO: 37] | Proteins in AAVhu68 Capsid | |
| Deamidated Residue + 1 | Broad Range of | Narrow |
| (Neighboring AA) | Percentages (%) | Ranges (%) |
| N57 | 78 to 100% | 80 to 100, 85 to 97 |
| (N-G) | | |
| N66 | 0 to 5 | 0, 1 to 5 |
| (N-E) | | |
| N94 | 0 to 15, | 0, 1 to 15, 5 to 12, 8 |
| (N-H) | | |
| N113 | 0 to 2 | 0, 1 to 2 |
| (N-L) | | |
| ~N253 | 10 to 25 | 15 to 22 |
| (N-N) | | |
| Q259 | 8 to 42 | 10 to 40, 20 to 35 |
| (Q-I) | | |
| ~N270 | 12 to 30 | 15 to 28 |
| (N-D) | | |
| ~N304 | 0 to 5 | 1 to 4 |
| (N-N) (position 303 also | | |
| N) | | |
| N319 | 0 to 5 | 0, 1 to 5, 1 to 3 |
| (N-I) | | |
| N329 * | 65 to 100 | 70 to 95, 85 to 95, |
| (N-G)*(position 328 | | 80 to 100, 85 to 100, |
| also N) | | |
| N336 | 0 to 100 | 0, 1 to 10, 25 to 100, |
| (N-N) | | 30 to 100, 30 to 95 |
| ~N409 | 15 to 30 | 20 to 25 |
| (N-N) | | |
| N452 | 75 to 100 | 80 to 100, 90 to 100, |
| (N-G) | | 95 to 100, |
| N477 | 0 to 8 | 0, 1 to 5 |
| (N-Y) | | |
| N512 | 65 to 100 | 70 to 95, 85 to 95, |
| (N-G) | | 80 to 100, 85 to 100, |
| ~N515 | 0 to 25 | 0, 1 to 10, 5 to 25, |
| (N-S) | | 15 to 25 |
| ~Q599 | 1 to 20 | 2 to 20, 5 to 15 |
| (Asn-Q-Gly) | | |
| N628 | 0 to 10 | 0, 1 to 10, 2 to 8 |
| (N-F) | | |
| N651 | 0 to 3 | 0, 1 to 3 |
| (N-T) | | |
| N663 | 0 to 5 | 0, 1 to 5, 2 to 4 |
| (N-K) | | |
| N709 | 0 to 25 | 0, 1 to 22, 15 to 25 |
| (N-N) | | |
| N735 | 0 to 40 | 0.1 to 35, 5 to 50, |
| | | 20 to 35 |

In certain embodiments, the AAVhu68 capsid is characterized, by having, capsid proteins in which at least 45% of N residues are deamidated at least one of positions N57, N329, N452, and/or N512 based on the numbering of amino acid sequence of SEQ ID NO: 37. In certain embodiments, at least about 60%, at least about 70%, at least about 80%, or at least 90% of the N residues at one or more of these N-G positions (i.e., N57, N329, N452, and/or N512, based on the numbering of amino acid sequence of SEQ ID NO: 37) are deamidated. In these and other embodiments, an AAVhu68 capsid is further characterized by having a population of proteins in which about 1% to about 20% of the N residues have deamidations at one or more of positions: N94, N253, N270, N304, N409, N477, and/or Q599, based on the numbering of amino acid sequence of SEQ ID NO: 37. In certain embodiments, the AAVhu68 comprises at least a subpopulation of vp1, vp2 and/or vp3 proteins which are deamidated at one or more of positions N35, N57, N66, N94, N113, N252, N253, Q259, N270, N303, N304, N305, N319, N328, N329, N336, N409, N410, N452, N477, N515, N598, Q599, N628, N651, N663, N709, N735, based on the numbering of amino acid sequence of SEQ ID NO: 37, or combinations thereof. In certain embodiments, the capsid proteins may have one or more amidated amino acids.

In another embodiment a recombinant adeno-associated virus (rAAV) is provided that has an AAVhu68 capsid and a vector genome, wherein (a) the AAV hu68 capsid comprises a heterogenous population of AAVhu68 vp1 proteins, a heterogenous population of AAVhu68 vp2 proteins; and a heterogenous population of AAVhu68 vp3 proteins, wherein the heterogenous AAVhu68 vp1, AAVhu68 vp2 and AAVhu68 vp3 proteins contain subpopulations with amino acid modifications comprising 50% to 100% deamidation in at least two asparagines (N) in asparagine-glycine pairs in two or more of N57, N329, N452, N512 of SEQ ID NO: 37 as determined using mass spectrometry and optionally further comprising subpopulations comprising other deamidated amino acids, wherein the deamidation results in an amino acid change, wherein the deamidated asparagines are deamidated to aspartic acid, isoaspartic acid, an interconverting aspartic acid/isoaspartic acid pair, or combinations thereof, wherein the AAVhu68 capsid further comprises subpopulations having one or more of:
(a) at least 65% of asparagines (N) in asparagine-glycine pairs located at positions N57 of the vp1 proteins are deamidated, based on the numbering of SEQ ID NO: 37;
(b) at least 75% of N in asparagine-glycine pairs in position N329 of the vp1, v2 and vp3 proteins are deamidated, based on the residue numbering of the amino acid sequence of SEQ ID NO: 37,
(c) at least 50% of N in asparagine-glycine pairs in position N452 of the vp1, v2 and vp3 proteins are deamidated, based on the residue numbering of the amino acid sequence of SEQ ID NO: 37; and/or
(d) at least 75% of N in asparagine-glycine pairs in position N512 of the vp1, v2 and vp3 proteins are deamidated, based on the residue numbering of the amino acid sequence of SEQ ID NO: 37, and
   a vector genome in the AAVhu68 capsid, the vector genome comprising a nucleic acid molecule comprising AAV inverted terminal repeat sequences and a non-AAV nucleic acid sequence encoding a PTH fusion as described herein operably linked to sequences which direct expression of PTH fusion in a target cell.

In one aspect, a recombinant AAV (rAAV) is provided. The rAAV includes an AAV capsid from adeno-associated virus rh91, and a vector genome packaged in the AAV capsid, said vector genome comprising AAV inverted terminal repeats (ITRs), a coding sequence for the canine proinsulin-serum albumin fusion of SEQ ID NO: 1, and regulatory sequences which direct expression of the canine proinsulin fusion.

In another embodiment, the rAAV includes an AAV capsid from adeno-associated virus rh91, and a vector genome packaged in the AAV capsid, said vector genome comprising AAV inverted terminal repeats (ITRs), a coding sequence for the canine proinsulin-transferrin fusion of SEQ ID NO: 3, and regulatory sequences which direct expression of the canine proinsulin fusion.

In another embodiment, the rAAV includes an AAV capsid from adeno-associated virus rh91, and a vector genome packaged in the AAV capsid, said vector genome comprising AAV inverted terminal repeats (ITRs), a coding sequence for the feline proinsulin-serum albumin fusion of SEQ ID NO: 32, and regulatory sequences which direct expression of the feline proinsulin fusion.

In one aspect, a recombinant AAV (rAAV) is provided. The rAAV includes an AAV capsid from adeno-associated virus hu68, and a vector genome packaged in the AAV capsid, said vector genome comprising AAV inverted terminal repeats (ITRs), a coding sequence for the canine proinsulin-serum albumin fusion of SEQ ID NO: 1, and regulatory sequences which direct expression of the canine proinsulin fusion.

In another embodiment, the rAAV includes an AAV capsid from adeno-associated virus hu68, and a vector genome packaged in the AAV capsid, said vector genome comprising AAV inverted terminal repeats (ITRs), a coding sequence for the canine proinsulin-transferrin fusion of SEQ ID NO: 3, and regulatory sequences which direct expression of the canine proinsulin fusion.

In another embodiment, the rAAV includes an AAV capsid from adeno-associated virus hu68, and a vector genome packaged in the AAV capsid, said vector genome comprising AAV inverted terminal repeats (ITRs), a coding sequence for the feline proinsulin-serum albumin fusion of SEQ ID NO: 32, and regulatory sequences which direct expression of the feline proinsulin fusion.

In one embodiment, the nucleic acid sequences encoding the proinsulin fusion constructs described herein are engineered into any suitable genetic element, e.g., naked DNA, phage, transposon, cosmid, RNA molecule (e.g., mRNA), episome, etc., which transfers the proinsulin fusion sequences carried thereon to a host cell, e.g., for generating nanoparticles carrying DNA or RNA, virions in a packaging host cell and/or for delivery to a host cell in a subject. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

In some embodiments, the proinsulin fusion constructs described herein may be delivered via virions other than rAAV. Such other virions may include any virus suitable for gene therapy may be used, including but not limited to adenovirus; herpes virus; lentivirus; retrovirus; etc. Suitably, where one of these other virions is generated, it is produced as a replication-defective virion.

A “replication-defective virus” or “virion” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the virion does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

### Expression Cassette

In some embodiments, the expression cassette refers to a nucleic acid molecule which comprises the proinsulin fusion construct coding sequences, promoter, and may include other regulatory sequences therefor. The expression cassette may be engineered into a genetic element and/or packaged into the capsid of a virion (e.g., a viral particle). Typically, such an expression cassette for generating a virion contains the proinsulin fusion construct sequences described herein flanked by packaging signals of the viral genome and other expression control sequences such as those described herein. Any of the expression control sequences can be optimized for a specific species using techniques known in the art including, e.g., codon optimization, as described herein.

The expression cassette typically contains a promoter sequence as part of the expression control sequences. In one embodiment, a constitutive promoter is used. In the plasmids and expression vectors described herein, a CB7 promoter may be used. CB7 is a chicken β-actin promoter with cytomegalovirus enhancer elements. Alternatively, liver-specific promoters may be used, including but not limited to alpha 1 anti-trypsin (A1AT), human albumin (Miyatake et al., J. Virol. 71:5124 32 (1997)) [humAlb], hepatitis B virus core promoter (Sandig et al., Gene Ther. 3:1002 9 (1996)), a TTR minimal enhancer/promoter, alpha-antitrypsin promoter, or liver-specific promoter (LSP) (Wu et al. Mol Ther. 16:280-289 (2008)). In one embodiment, the liver-specific promoter thyroxin binding globulin (TBG) is used. Other promoters, such as viral promoters, constitutive promoters, regulatable promoters (see, e.g., WO 2011/126808 and WO 2013/04943), or a promoter responsive to physiologic cues may be used may be utilized in the expression vectors described herein.

In one embodiment, an expression cassette comprises a CB7 promoter, a chicken beta actin intron, a canine mutant pre-insulin with a canine insulin leader fused via a linker to a feline albumin, and a rabbit beta globin poly is provided, e.g., in SEQ ID NO: 26. SEQ ID NO: 27 illustrates a vector genome comprising a shortened AAV 5′ ITR, spacer sequences, SEQ ID NO: 27, spacer sequences, and a shortened AAV 3′ ITR.

In another embodiment, an expression cassette comprises a CB7 promoter, a chicken beta actin intron, a canine mutant pre-insulin with a canine insulin leader fused via a linker to a feline transferrin, and a rabbit beta globin poly is provided, e.g., in SEQ ID NO: 28. SEQ ID NO: 29 illustrates a vector genome comprising a shortened AAV 5′ ITR, spacer sequences, SEQ ID NO: 28, spacer sequences, and a shortened AAV 3′ ITR.

In another embodiment, an inducible promoter is used. An example of an inducible promoter useful herein includes that described in International Patent Application No. PCT/US2021/043219, entitled “Canine and Feline Inducible Expression Constructs for Gene Therapy Applications”, published Feb. 3, 2022, which is incorporated herein by reference. Briefly, the inducible promoter comprises a promoter; an activation domain comprising a canine or feline transactivation domain and a FKBP12-rapamycin binding (FRB) domain of canine or feline FKBP12-rapamycin-associated protein (FRAP); a DNA binding domain comprising a zinc finger homeodomain (ZFHD) and one, two or three FK506 binding protein domain (FKBP) subunit genes; and at least 8 copies of the binding site for ZFHD (8×ZFHD) followed by a minimal IL2 promoter. The presence of an effective amount of a rapamycin or a rapalog induces expression of the transgene in a host cell.

In addition to a promoter, an expression cassette and/or an expression vector may contain other appropriate transcription initiation, termination, enhancer sequences, efficient RNA processing signals such as splicing and polyadenylation (poly A) signals; sequences that stabilize cytoplasmic mRNA; sequences that enhance translation efficiency (i.e., Kozak consensus sequence); sequences that enhance protein stability; and when desired, sequences that enhance secretion of the encoded product. Illustrative examples of suitable polyA sequences include, e.g., rabbit beta globin, SV40, bovine growth hormone (bGH), and TK poly A.

Illustrative examples of suitable enhancers include, e.g., the alpha fetoprotein enhancer, the TTR minimal promoter/enhancer, LSP (TH-binding globulin promoter/alpha1-microglobulin/bikunin enhancer), amongst others. In one embodiment, the polyA is a rabbit globin poly A. In one embodiment, the poly A has the sequence of SEQ ID NO: 20.

These control sequences are “operably linked” to the proinsulin fusion construct sequences. As used herein, the term “operably linked” refers to both expression control sequences that are contiguous with the gene of interest and expression control sequences that act in trans or at a distance to control the gene of interest.

In one embodiment, a rAAV is provided which includes a 5′ ITR, CB7 promoter, chicken beta-actin intron, coding sequence for the protein of SEQ ID NO: 1, a rabbit globin poly A, and a 3′ ITR. In one embodiment, a rAAV is provided which includes a 5′ ITR, CB7 promoter, chicken beta-actin intron, coding sequence for the protein of SEQ ID NO: 3, a rabbit globin poly A, and a 3′ ITR. In one embodiment, a rAAV is provided which includes a 5′ITR, CB7 promoter, chicken beta-actin intron, coding sequence for the protein of SEQ ID NO: 3, a rabbit globin poly A, and a 3′ ITR.

The minimal sequences required to package the expression cassette into an AAV viral particle are the AAV 5′ and 3′ ITRs, which may be of the same AAV origin as the capsid, or of a different AAV origin (to produce an AAV pseudotype).

For packaging an expression cassette into an AAV capsid to form virions (i.e., an rAAV vector or rAAV particle), the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV virion. For example, as described above, a pseudotyped AAV may contain ITRs from a source which differs from the source of the AAV capsid. In one embodiment, a chimeric AAV capsid may be utilized. Still other AAV components may be selected. Sources of such AAV sequences are described herein and may also be isolated or obtained from academic, commercial, or public sources (e.g., the American Type Culture Collection, Manassas, VA). The AAV sequences may be obtained through synthetic or other suitable means by reference to published sequences such as are available in the literature or in databases such as, e.g., GenBank, PubMed, or the like.

Methods for generating and isolating AAV virions suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In another system, the transgene flanked by ITRs and rep/cap genes are introduced into insect cells by infection with baculovirus-based expression vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in their entirety. Methods of making and using these and other AAV production systems are also described in the following US patents, the contents of each of which is incorporated herein by reference in their entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, Adv. Biochem. Engin/Biotechnol. 99:119-145; Buning et al., 2008, J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques.

Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

The rAAV described herein comprise a selected capsid with a vector genome packaged inside. The vector genome (or rAAV genome) comprises 5′ and 3′ AAV inverted terminal repeats (ITRs), the polynucleotide sequence encoding the insulin protein, and regulatory sequences which direct insertion of the polynucleotide sequence encoding the insulin protein to the genome of a host cell.

Methods of Treating Subjects with the Disclosed Compositions

Also provided are compositions which include the virion constructs described herein. The pharmaceutical compositions described herein are designed for delivery to canine or feline subjects in need thereof by any suitable route or a combination of different routes. Direct delivery to the liver (optionally via intravenous, via the hepatic artery, or by transplant), direct delivery to the pancreas, oral, inhalation, intranasal, intratracheal, intraarterial, intraocular, intravenous, intramuscular, subcutaneous, intradermal, and other parental routes of administration. The virions described herein may be delivered in a single composition or multiple compositions. Optionally, two or more different AAV may be delivered, or multiple viruses [see, e.g., WO 2011/126808 and WO 2013/049493].

In some embodiments, the pharmaceutical compositions described herein are designed for delivery to canine or feline subjects in need thereof by intramuscular administration.

In another embodiment, multiple viruses may contain different replication-defective viruses (e.g., AAV and adenovirus). In one embodiment, administration is intramuscular. In another embodiment, administration is intravenous.

In some embodiments, a course of treatment may involve repeat administration of the same virion (e.g., an AAVrh91 virion) or a different virion (e.g., an AAVrh91 and an AAV3B.AR2.12). Still other combinations may be selected using the virions described herein. In some embodiments, the composition described herein may be combined in a regimen involving other diabetic drugs or protein-based therapies (including e.g., insulin analogues, insulin, oral antihyperglycemic drugs (sulfonylureas, biguanides, thiazolidinediones, and alpha-glucosidase inhibitors). In some embodiments, the composition described herein may be combined in a regimen involving lifestyle changes including dietary and exercise regimens.

As used herein the terms “proinsulin construct”, “proinsulin expression construct” and synonyms include the proinsulin sequence as described herein in combination with a leader (whether native or heterologous). The terms “proinsulin construct”, “proinsulin expression construct” and synonyms can be used to refer to the nucleic acid sequences encoding the proinsulin fusion protein or the expression products thereof. In this specification, the proinsulin sequence with the leader is also referred to as a “pre-proinsulin”. In certain embodiments, the “proinsulin construct” may also encompass the fusion proteins described herein.

The replication-defective viruses can be formulated with a physiologically acceptable carrier for use in gene transfer and gene therapy applications. In the case of AAV virions, quantification of the genome copies (“GC” or “gc”) may be used as the measure of the dose contained in the formulation. Any method known in the art can be used to determine the genome copy (GC) number of the replication-defective virus compositions of the invention. One method for performing AAV GC number titration is as follows: purified AAV vector genome samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies is the quantitative-PCR (qPCR), particularly the optimized qPCR or digital droplet PCR.

Also, the replication-defective virus compositions can be formulated in dosage units to contain an amount of replication-defective virus that is in the range of about 1.0×10⁹ GC to about 1.0×10¹⁵ GC. In another embodiment, this amount of viral genome may be delivered in split doses. In one embodiment, the dose is about 1.0×10¹⁰ GC to about 3.0×10¹³ GC for an average canine or feline subject of about 5-10 kg. In another embodiment, the dose is about 1×10⁹ GC. For example, the dose of AAV virus may be about 1×10¹⁰ GC, 1×10¹¹ GC, about 5×10¹¹ GC, about 1×10¹² GC, about 5×10¹² GC, or about 1×10¹³ GC. In another embodiment, the dosage is about 1.0×10⁹ GC/kg to about 3.0×10¹³ GC/kg body mass for a canine or feline subject. In another embodiment, the dose about 1×10⁹ GC/kg. In another embodiment, the dose about 1×10¹⁰ GC/kg. In another embodiment, the dose about 1×10¹¹ GC/kg. For example, the dose of AAV virus may be about 1×10¹⁰ GC/kg, 1×10¹¹ GC/kg, about 5×10¹¹ GC/kg, about 1×10¹² GC/kg, about 5×10¹² GC/kg, or about 1×10¹³ GC/kg.

In another aspect, a method of sustained expression of a proinsulin fusion protein in a canine or feline subject is provided. The method includes administering a composition as described herein to a subject in need thereof. In one embodiment, the composition includes a virion containing a proinsulin-serum albumin fusion protein expression cassette, as described herein. In some embodiments, the method provided herein results in expression of the proinsulin fusion protein in the subject for at least one week, at least two weeks, at least four weeks, at least 6 weeks, at least 8 weeks, at least 10 weeks, at least 12 weeks, at least 16 weeks, at least 20 weeks, at least 30 weeks, at least 40 weeks, at least 50 weeks, or at least 60 weeks. In some embodiments, the method provided herein results in expression of the proinsulin fusion protein in the subject for at least eight weeks. In some embodiments, the method provided herein results in expression of the proinsulin fusion protein in the subject at a therapeutically effective concentration for at least one month, at least three months, at least six months, or at least twelve months.

In one embodiment, the constructs may be delivered in volumes from 1 μL to about 100 mL for a veterinary subject. See, e.g., Diehl et al, J. Applied Toxicology, 21:15-23 (2001) for a discussion of good practices for administration of substances to various veterinary animals. This document is incorporated herein by reference. As used herein, the term “dosage” can refer to the total dosage delivered to the subject in the course of treatment, or the amount delivered in a single (of multiple) administration.

In one embodiment, the composition is administered in combination with an effective amount of insulin. Various commercially available insulin products are known in the art, including, without limitation, protamine zinc recombinant human insulin (ProZinc®), porcine insulin zinc suspension (Vetsulin®), and insulin glargine (Lantus®). In some embodiments, combination of the rAAV described herein with insulin decreases insulin dose requirements in the subject, as compared to prior to treatment with the AAV virion. Such dose requirements may be reduced by 10% or more, 20% or more, 30% or more, 40% or more, 50% or more, 60% or more, 70% or more, 80% or more, or 90% or more. The treating physician may determine the correct dosage of insulin needed by the subject. For example, the subject may be being treated using insulin or other therapy, which the treating physician may continue upon administration of the AAV virion. Such insulin or other co-therapy may be continued, reduced, or discontinued as needed subsequently.

In one embodiment, a composition comprising the expression cassette, vector genome, rAAV, or other composition described herein for gene therapy is delivered as a single dose per subject. In one embodiment, the subject is delivered a therapeutically effective amount of a composition described herein. As used herein, a “therapeutically effective amount” refers to the amount of the expression cassette or virion, or a combination thereof that delivers and expresses in the target cells an amount of proinsulin-serum albumin sufficient to reach the therapeutic goal. In certain embodiments, the therapeutic goal is to ameliorate or treat one or more of the symptoms of type I diabetes, type II diabetes or metabolic syndrome. A therapeutically effective amount may be determined based on an animal model, rather than a canine or feline subject. In another embodiment, the therapeutic goal is remission of the metabolic disease in the subject.

The above-described recombinant AAV virions may be delivered to host cells according to published methods. The rAAV, preferably suspended in a physiologically compatible carrier, may be administered to a desired subject including a canine. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

In some embodiments, the compositions of the invention may contain, in addition to the rAAV and/or variants and carrier(s), other conventional pharmaceutical ingredients, such as preservatives, or chemical stabilizers. Illustrative preservatives include chlorobutanol, potassium sorbate, sorbic acid, sulfur dioxide, propyl gallate, the parabens, ethyl vanillin, glycerin, phenol, and parachlorophenol. Illustrative chemical stabilizers include gelatin and albumin.

The recombinant virions and constructs described herein may be used in preparing a medicament for delivering a proinsulin fusion protein construct to a subject in need thereof, supplying insulin having an increased half-life to a subject, and/or for treating type I diabetes, type II diabetes, or metabolic syndrome in a subject.

In one aspect, a method of treating diabetes is provided. The method includes administering a composition as described herein to a canine or feline subject in need thereof. In one embodiment, the composition includes a virion containing a proinsulin fusion protein expression cassette, as described herein.

In another embodiment, a method for treating type 2 diabetes in a canine or feline is provided. The method includes administering a virion comprising a nucleic acid molecule comprising a sequence encoding a proinsulin fusion protein as described herein.

In another embodiment, a method for treating type 1 diabetes in a canine or feline is provided. The method includes administering a virion comprising a nucleic acid molecule comprising a sequence encoding a proinsulin fusion protein as described herein.

In another aspect, a method of treating a metabolic disease in a canine or feline is provided. The method includes administering a composition as described herein to a canine or feline subject in need thereof. In one embodiment, the composition includes a virion containing a proinsulin fusion protein expression cassette, as described herein. In one embodiment, the metabolic disease is Type I diabetes. In one embodiment, the metabolic disease is Type II diabetes. In one embodiment, the metabolic disease is metabolic syndrome.

In another embodiment, a method for treating diabetes in a canine or feline is provided. The method includes administering a virion comprising a nucleic acid molecule comprising a sequence encoding a proinsulin-serum albumin fusion protein as described herein, wherein the virion is administered after insulin is administered in a subject. In some embodiments, the virion is administered at least 1 day, at least 3 days, at least 5 days, at least one week, at least 2 weeks, at least 3 weeks, or at least 4 weeks after insulin is administered to a subject.

In another embodiment, a method for preventing cataract formation in diabetic canines or felines is provided. The method includes administering a virion comprising a nucleic acid molecule comprising a sequence encoding a proinsulin-serum albumin fusion protein as described herein. In some embodiments, the virion is administered after insulin is administered to a subject.

In another embodiment, a method for reducing the blood glucose concentration in diabetic canines or felines is provided. The method includes administering a virion comprising a nucleic acid molecule comprising a sequence encoding a proinsulin-serum albumin fusion protein as described herein.

As used herein, the term “treatment” or “treating” is defined encompassing administering to a subject one or more compounds or compositions described herein for the purposes of amelioration of one or more symptoms of type I diabetes, type II diabetes (T2DM) or metabolic syndrome. “Treatment” can thus include one or more of reducing progression of type I diabetes, type II diabetes or metabolic syndrome, reducing the severity of the symptoms, removing the disease symptoms, delaying progression of disease, or increasing efficacy of therapy in a given subject.

As used herein, the term “remission” refers to the ability to cease insulin treatment when the cat or dog no longer exhibits clinical signs of diabetes and has normal blood glucose levels.

In another embodiment, a method for treating T2DM in a feline or canine is provided. The method includes administering a virion comprising a nucleic acid molecule comprising a sequence encoding a fusion protein as described herein.

In another aspect, a method of treating a metabolic disease in a feline or canine is provided. The method includes administering a composition as described herein to a feline or canine subject in need thereof. In one embodiment, the composition includes a virion containing a proinsulin fusion protein expression cassette, as described herein.

In another aspect, a method of reducing fasting blood sugar in a canine or feline subject is provided. The method includes administering a composition as described herein to a subject in need thereof. In one embodiment, the composition includes a virion containing a proinsulin-serum albumin fusion protein expression cassette, as described herein. In some embodiments, the method provided herein decreases fasting blood glucose in the subject by at least about 10%, at least about 15%, at least about 20%, at least about 25%, at least about 30%, at least about 35%, at least about 40%, at least about 45%, or at least about 50%. In some embodiments, the method provided herein decreases fasting blood glucose in the subject by about 20%. In some embodiments, the method provided herein decreases fasting blood glucose in the subject by about 30%. In some embodiments, the method provided herein decreases fasting blood glucose in the subject by about 40%.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. In some embodiments, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. In some embodiments, BLAST and BLAST 2.0 algorithms and the default parameters are used.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequences. A suitable amino acid fragment may be at least about 8 amino acids in length and may be up to about 150 amino acids. Generally, when referring to “identity”, “homology”, or “similarity” between two different sequences, “identity”, “homology” or “similarity” is determined in reference to “aligned” sequences.

“Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs.

The term “amino acid substitution” and its synonyms are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In an embodiment, the insulin-serum albumin fusion is caninized or felinized.

By “caninized” is meant that the fusion protein comprises an amino acid sequence that is compatible with canine, such that the amino acid sequence is unlikely to be seen as foreign by the immune system of a canine subject. In the present disclosure, the term for a polypeptide preceded by the prefix “ca” refers to a variant of the human polypeptide in which the human fusion domain is replaced with the canine homolog of that fusion domain and, where the proinsulin is a fragment or variant of a human protein, the proinsulin is replaced with the canine homolog of that fragment or variant.

By “felinized” is meant that the fusion protein comprises an amino acid sequence that is compatible with feline, such that the amino acid sequence is unlikely to be seen as foreign by the immune system of a feline subject. In the present disclosure, the term for a polypeptide preceded by the prefix “fe” refers to a variant of the human polypeptide in which the human fusion domain is replaced with the feline homolog of that fusion domain and, where the proinsulin is a fragment or variant of a human protein, the proinsulin is replaced with the feline homolog of that fragment or variant.

As noted elsewhere herein, the present disclosure extends to fusion proteins that are compatible with species other than canine or feline. In this context, the fusion proteins can be referred to as “speciesized”, referring to the target species to which the molecule will be administered.

In some embodiments, the compositions and methods described herein are intended to be for use in feline animals. The term feline (family Felidae) refers to any of 37 cat species that among others include the cheetah, puma, jaguar, leopard, lion, lynx, tiger, and domestic cat. In an embodiment, the subject is a domestic cat. In some embodiments, the compositions and methods described herein are intended to be for use in canine animals. The term canine refers to any of species found in the Canidae family that among others includes domestic dogs, wolves, and foxes. In an embodiment, the subject is a domestic dog, also known as Canis lupus familiaris or Canis familiaris.

As used in the description of the invention and the appended claims, the singular forms “a”, “an”, and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (or).

As used herein, the phrase “consisting essentially of” refers to the genera or species of active pharmaceutical agents recited in a method or composition, and further can include other agents that, on their own do not have substantial activity for the recited indication or purpose.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

The term “regulation” or variations thereof as used herein refers to the ability of a composition to inhibit one or more components of a biological pathway.

As used herein, “disease”, “disorder” and “condition” are used interchangeably, to indicate an abnormal state in a subject.

The terms “subject,” “individual,” and “patient” interchangeably refer to a mammal, a human or a non-human primate, domesticated mammals (e.g., canine or feline), laboratory mammals, and agricultural mammals. In various embodiments, the subject can be a human (e.g., adult male, adult female, adolescent male, adolescent female, male child, female child). In various embodiments, the subject is a companion animal. Illustrative companion animals include, but are not limited to, dogs, cats, horses, rabbits, ferrets, birds, and guinea pigs. In various embodiments, the subject is a canine. In various embodiments, the subject is a feline. In various embodiments, the subject is a mammal.

The terms “viral particle” or “virion” as used herein refers a macromolecular complex capable of delivering a foreign nucleic acid molecule into a cell independent of another agent. Independent particles of viruses, “viral particle” or “virion”, consist of genetic material, (i.e., DNA or RNA that encode the transgenes) and the capsid. A particle can be a viral particle or non-viral particle. Viral particle includes retroviral particle, lentiviral particle, and Adeno-associated viral particle. The term “adeno-associated viral particle” may be used interchangeably with “recombinant adeno-associated virus (rAAV) vector” and indicates the presence of a nuclease-resistant AAV capsid having packaged therein a replication-defective vector genome. Non-viral particles are limited to liposomes, nanoparticles, and other encapsulation systems for delivery of polynucleotides into cells.

The term “transgene” refers to the transferred nucleic acid itself. The transgene may be a naked nucleic acid molecule (such as a plasmid) or RNA. The transgene may include a polynucleotide encoding one or more polypeptides (e.g., an insulin fusion protein). The transgene may include a polynucleotide encoding one or more heterologous protein (e.g., an insulin fusion protein), one or more capsid proteins, and other proteins necessary for transduction of the polynucleotide into a target cell.

The term “transduce” refers to introduction of a nucleic acid into a cell or host organism by way of a particle (e.g., an Adeno-associated viral particle). Introduction of a transgene into a cell by a viral particle can therefore be referred to as “transduction” of the cell. The transgene may or may not be integrated into the genomic nucleic acid of a transduced cell. If an introduced transgene becomes integrated into the nucleic acid (genomic DNA) of the recipient cell or organism it can be stably maintained in that cell. Alternatively, the introduced transgene may exist in the recipient cell or host organism extra-chromosomally, or only transiently. A “transduced cell” is therefore a cell into which the transgene has been introduced by way of transduction. Thus, a “transduced” cell is a cell into which, a polynucleotide has been introduced.

As used herein, the term “host cell” may refer to the packaging cell line in which a virion (e.g., a recombinant AAV or rAAV) is produced from a production plasmid. In the alternative, the term “host cell” may refer to any target cell in which expression of a gene product described herein is desired. Thus, a “host cell,” refers to a prokaryotic or eukaryotic cell (e.g., bacterial cell, human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the virion or recombinant virus. In a further embodiment, the term “host cell” is an intestine cell, a small intestine cell, a pancreatic cell, a liver cell.

As used herein, the term “target cell” refers to any target cell in which expression of a heterologous nucleic acid sequence or protein is desired. In certain embodiments, the target cell is a liver cell. In some embodiments, the target cell is a muscle cell.

As used herein, an “expression cassette” or “vector genome” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences (also referred to as elements) that are contiguous or non-contiguous with the nucleic acid sequence and regulatory sequences that act in trans or cis nucleic acid sequence. Such regulatory sequences typically include, e.g., one or more of a promoter, an enhancer, a transcription factor, transcription terminator, an intron, sequences that enhance translation efficiency (i.e., a Kozak consensus sequence), efficient RNA processing signals such as slicing and a polyadenylation sequence, sequences that stabilize cytoplasmic mRNA, for example Woodchuck Hepatitis Virus (WHP) posttranslational Regulatory Element (WPRE), and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequence, i.e., 5′-untranslated regions (5′ UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette can be used for generating a virion and contains the coding sequence for the gene product described herein flanked by packaging signals of the viral genome and other expression control sequences such as those described herein. In certain embodiments, a vector genome may contain two or more expression cassettes.

As used herein, “administering” refers to local and systemic administration, e.g., including enteral, parenteral, pulmonary, and topical/transdermal administration. Routes of administration for pharmaceutical ingredients (e.g., virions) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial.

The terms “systemic administration” and “systemically administered” refer to a method of administering a pharmaceutical ingredient or composition to a mammal so that the pharmaceutical ingredient or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “effective amount” or “pharmaceutically effective amount” refer to the amount and/or dosage, and/or dosage regime of one or more pharmaceutical ingredients (e.g., virions) necessary to bring about the desired result.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In some embodiments, the reduction or elimination of one or more symptoms of pathology or disease can include, e.g., measurable and sustained decrease of fasting blood glucose.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what is regarding as the invention.

### Example 1: Generation of a Half-Life Extended Canine Insulin-Serum Albumin Fusion and Evaluation of In Vitro Potency of Canine Insulin Fusion Proteins

A canine insulin-serum albumin fusion protein designed to be administered via an AAV virion has been developed for the management of hyperglycemia and hyperglycemia-associated clinical signs of diabetes mellitus in dogs.

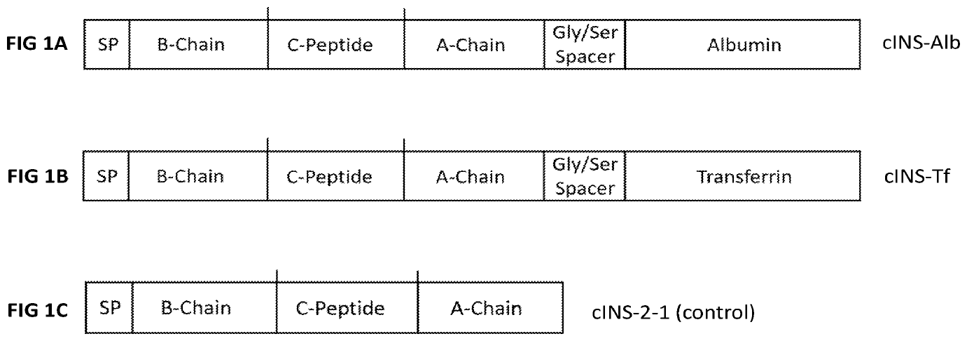

A canine preproinsulin-serum albumin fusion protein (cINS-Alb) was generated by constructing a fusion polypeptide containing the following elements (FIG. 1A) (SEQ ID NO: 1):
Native canine insulin signal peptide (SP)
Canine proinsulin, where the native sequence has been modified at 3 amino acid positions (K29R, R31K and L62R) to incorporate 2 furin cleavage sites at existing protease cleavage sites (B-Chain, C-Peptide, A-Chain)
14 aa Gly/Ser linker comprising the sequence GGGGGGGGSGGGS (SEQ ID NO: 8)
Canine serum albumin

A canine preproinsulin fusion protein was made with the same elements as above except the canine serum albumin sequence was replaced with canine transferrin (cINS-Tf) (FIG. 1B) (SEQ ID NO: 3). The expression cassette for the albumin fusion is provided in SEQ ID NO: 26 and the vector genome of the production plasmid is in SEQ ID NO: 27. A control canine preproinsulin sequence (no fusion), with the 3 amino acid modifications (K29R, R31K and L62R) was also generated in an AAV virion as a control (cINS-2-1) (FIG. 1C) (SEQ ID NO: 5; expression cassette for control is in SEQ ID NO: 30 and vector genome is in SEQ ID NO: 31). The vector genomes of the production plasmids all containing shortened AAV 5′ and 3′ ITRs. SEQ ID NO: 28 provides the expression cassette for a canine proinsulin-transferrin fusion protein. The vector genome is provided in SEQ ID NO: 29.

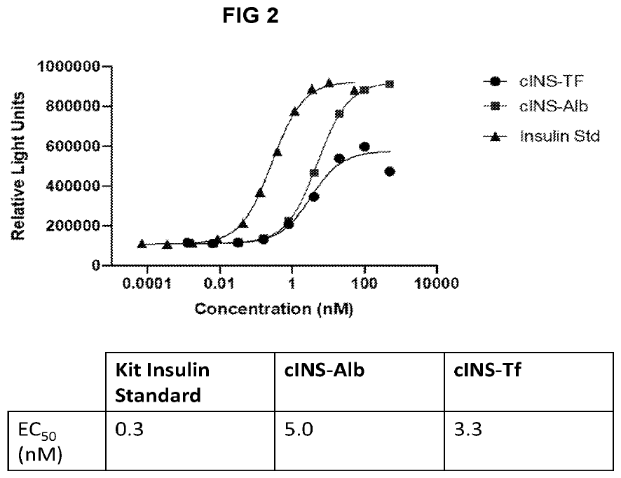

A study was conducted to evaluate the in vitro potency of the two fusion proteins (cINS-Alb and cINS-Tf), C-terminal histidine-tagged versions of each of the proteins was produced in mammalian cells following transient transfection of expression plasmids containing the cDNA encoding the respective proteins. The proteins were purified from the cell supernatant via nickel affinity chromatography and were assayed for insulin bioactivity using a PathHunterÒ™ Insulin Bioassay kit (Eurofins™ DiscoverX Products, LLC) as per the kit instructions. A standard insulin provided in the kit was used as a reference control. Both cINS-Alb and cINS-Tf demonstrated bioactivity in this assay (FIG. 2). The modifications made to the insulin molecule in both of the fusion proteins resulted in a small loss in potency as indicated by the shift in EC₅₀ values.

### Example 2: Generation of AAV Virions Comprising Canine Insulin Fusion Protein Transgenes and Evaluation of In Vivo Efficacy

To generate AAV virions, codon-optimized transgenes encoding a control with the 3 amino acid modification without a fusion partner (AAVrh91.cIns.2-1) and two insulin fusion proteins (SEQ ID NOs: 1, 3, and 5) were cloned into a plasmid containing a CB7 promoter element (comprising a hybrid cytomegalovirus enhancer and chicken b-actin promoter). The expression constructs were flanked by shortened 130 bp 5′ and 3′ AAV inverted terminal repeats (ITR) in the plasmid and were packaged in an AAV serotype rh91 capsid (AAVrh91) virion by triple transfection with a trans plasmid expressing the AAVrh91 capsid and a helper plasmid expressing adenoviral helper functions necessary for replication and production, which adenoviral helper functions are not provided by the HEK 293 packaging cells which express E1 adenoviral functions. During replication and packaging, the 5′ and 3′ ITRs revert to the full wild-type 145 bp ITR length. The AAV virions were subsequently purified by iodixanol gradient purification titred by Taqman™ quantitative PCR.

A study was conducted to evaluate the in vivo efficacy of three AAV-delivered canine insulin fusion proteins in a mouse model of diabetes. NOD-SCID mice that had been treated with streptozotocin to induce diabetes were allocated to one of four groups as shown below in Table 1. A 5^{th} group of non-diabetic NOD-SCID mice were included as a control.

TABLE 1
Study Groups

| | | STZ-induced | | Dose |
|---|---|---|---|---|
| Group | N | diabetes? | Vector Genome | (GC/mouse) |
| 1 | 7 | Yes | PBS | — |
| 2 | 8 | Yes | AAVrh91.cIns-Alb | 1 × 10¹¹ |
| 3 | 8 | Yes | AAVrh91.cIns-Tf | 1 × 10¹¹ |
| 4 | 8 | Yes | AAVrh91.cIns.2-1 (control) | 1 × 10¹¹ |
| 5 | 10 | No | PBS | — |

Mice were administered virion (1×10¹¹ GC/mouse) via intramuscular (IM) injection. Twice per week food was removed from the cages 6 hours prior to sampling for fasting blood glucose. Two mice from Group 2 were euthanized (at Day 45 and Day 52 respectively) due to hypoglycemia. One mouse from Group 3 was euthanized at Day 28 due to a poor body condition score and high blood glucose. Fasting blood glucose was measured by glucometer, which had an upper limit of 600 mg/dL.

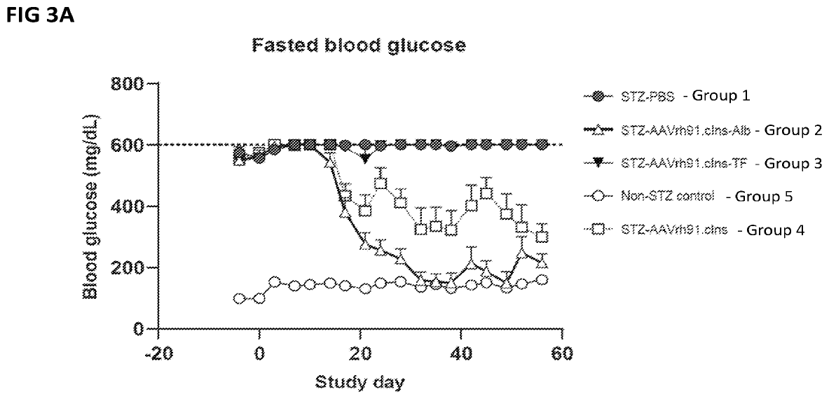

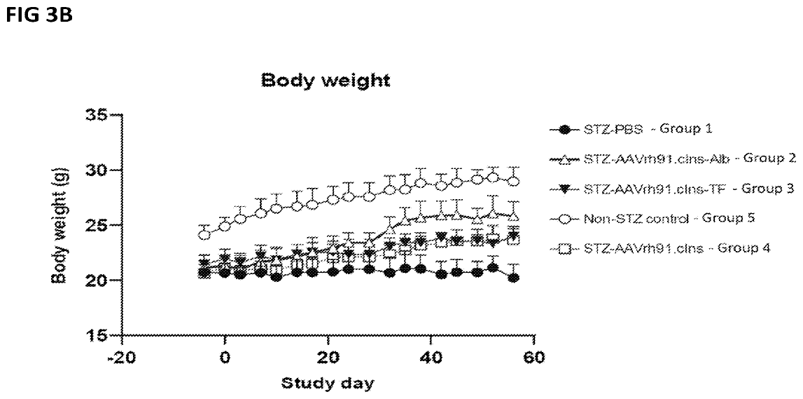

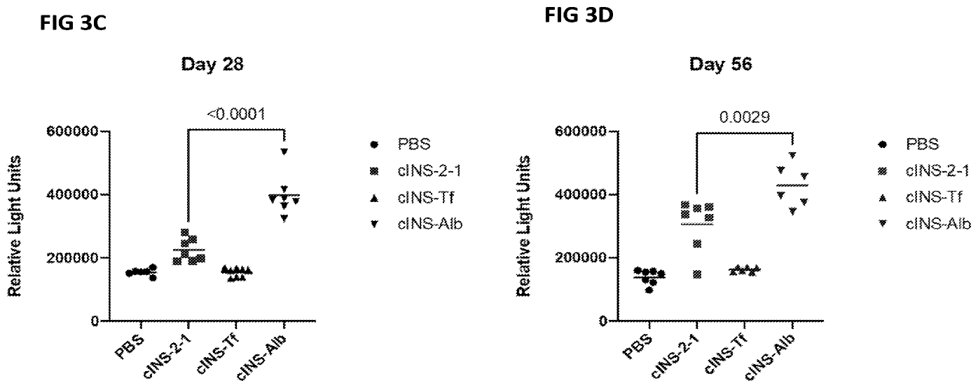

Animals receiving AAV virions containing the control cINS-2-1 (Group 4) and cINS-Alb (Group 2) transgenes had significant reduction in blood glucose levels compared to the control Group 1 (FIG. 3A). Mice that received the AAV containing the cINS-Alb fusion (Group 2) exhibited a more pronounced and quicker drop in fasting blood glucose levels than the AAV containing the control cINS-2-1 (Group 4). By Day 32 blood glucose levels in Group 2 had normalized and were similar to those observed in the non-diabetic cohort of mice (Group 5). In contrast to the significant reduction in blood glucose levels observed in animals that received the AAV containing the cINS-Alb fusion (Group 2), no decrease in blood glucose were observed in the cohort of animals administered the AAV containing the cINS-Tf fusion (Group 3).

The change in body weight of the mice is shown in FIG. 3B. Mice administered the AAV containing the cINS-Alb fusion tended to gain more weight than diabetic animals in the other groups, although it was not statistically significant.

To examine the relative biological potency of the expressed canine insulin proteins, serum from the mice taken at Day 28 and Day 56 was tested in the PathHunterÒ™ Insulin Bioassay kit as detailed above. As seen in FIG. 3C, at both Day 28 and Day 56, animals administered the AAV containing the cINS-Alb fusion had a significantly higher level of circulating, potent insulin than those in the other cohorts. Surprisingly, although the cINS-Tf protein showed moderate activity in the in vitro assay, no activity or evidence of efficacy of the insulin transferrin fusion protein was observed in vivo. This data shows that cINS-Alb exhibited higher bioactivity than cINS-Tf and AAV containing the cINS-Alb fusion exhibited significantly higher in vivo efficacy than AAV containing the cINS-Tf fusion.

### Example 3: Evaluation of AAV-caINS-Alb in Healthy Canine Subjects

A study will be conducted to evaluate the expression, immunogenicity, and effect on blood glucose following administration of an AAVrh91 virion expressing caINS-Alb in healthy dogs. Two groups of healthy dogs weighing approximately 10 kg to 15 kg will be administered an intramuscular dose of AAV-caINS-Alb at a dose of 1×10¹¹ or 1×10¹² GC/kg. Prior to administration of the AAV-caINS-Alb, a series of health screening measures including veterinary examinations, serum fructosamine analysis as well as hematology, clinical chemistry analysis, and urinalysis will be performed. Serum samples will then be collected weekly for up to 8 weeks to determine the level of circulating caINS-Alb protein using a sandwich ELISA protocol as described below.

Potential immunogenicity to caINS-Alb will be determined by measuring anti-caINS-Alb antibodies in a bridging assay. Blood glucose levels will be measured twice daily and a continuous glucose monitoring system (FreeStyle Libre™, Abbott) will be used to record interstitial glucose concentrations at regular intervals. Animals will be observed daily for signs of anaphylaxis, lethargy, and hypoglycemia.

To measure serum concentrations of caINS-Alb, a sandwich ELISA is used. ELISA plates are coated with 0.2 ug/ml of mouse anti-human insulin (clone 7F8, Life Technologies) and blocked with PBS/0.05% Tween 20/1% casein. Coated wells are incubated for 1 h at room temperature with 100 μL of appropriately diluted serum samples, diluted in PBS/0.05% Tween 20/1% casein. Purified caINS-Alb standards ranging from 200 ng/ml to 0.19 ng/ml are used to establish a standard curve. After washing, the plates are incubated for 1 h with a 1/2000 dilution of goat anti-dog albumin (Bethyl Laboratories) in PBS/0.05% Tween 20/1% casein. Plates are then washed five times with PBS/0.05% Tween 20 and developed by the addition of Tetramethylbenzidine (TMB) substrate. Development is stopped by the addition of 2M H₂SO₄ and absorbance is read at 450 nm.

### Example 4: Evaluation of AAV-caINS-Alb Efficacy in a Canine Streptozotocin (STZ) Induced Diabetes Model

The efficacy of a single administration of an AAVrh91 virion expressing caINS-Alb to adequately maintain blood glucose levels in diabetic dogs will be assessed in a non-randomized, non-blinded, laboratory study of six Beagle dogs with chemically induced diabetes mellitus that are on a stable diabetic treatment regimen.

A low dose (1×10¹¹ gc/kg, N=3) of AAVrh91 virion expressing caINS-Alb and a high dose (1×10¹² gc/kg, N=3) AAVrh91 virion expressing caINS-Alb will be evaluated. Prior to administration of the AAV-caINS-Alb, a series of health screening measures including veterinary examinations, serum fructosamine analysis as well as hematology, clinical chemistry analysis, and urinalysis will be performed. Throughout the study, 3× daily blood glucose measurements via glucometer and 2× daily clinical observations will be performed. On Day-1, subjects will be acclimated to placement of continuous glucose monitors and 3× daily interstitial glucose concentrations will be collected.

Over the course of study, in addition to the daily procedures initiated at baseline, blood collections for fructosamine analysis, hematology, and clinical chemistry will be performed every 14 days until Day 42 and once more on Day 63. Blood collections for determining circulating caINS-Alb protein and anti-caINS-Alb antibodies will be collected regularly throughout the study and body weights will be measured every 7 days. Required supplementary insulin dose levels, weight gain, and glycemic control will be used to evaluate the efficacy of the test product at the two doses tested in individual subjects.

### Example 5: Evaluation of AAV-caINS-Alb Efficacy in a Canine Diabetes Model

A study will be conducted to evaluate the efficacy of a single administration of an AAVrh91 virion expressing caINS-Alb to adequately maintain blood glucose levels in diabetic dogs.

Dogs with previously diagnosed diabetes mellitus on stable insulin treatment with moderate to good glycemic control will be treated with either placebo or AAV-caINS-Alb (administered intramuscularly). Newly diagnosed dogs with diabetes mellitus will be treated with either placebo or AAV-caINS-Alb (administered intramuscularly). Prior to administration of the AAV-caINS-Alb, a series of health screening measures including veterinary examinations, serum fructosamine analysis as well as hematology, clinical chemistry analysis, and urinalysis will be performed. Glycemic control will be assessed prior to AAV administration and throughout the study by measuring interstitial glucose concentrations continuously with a continuous glucose monitor or twice daily using a glucometer. Throughout the study dogs are fed a consistent diet and body weight is monitored. Over the course of study, in addition to the blood glucose monitoring, blood collections for fructosamine analysis, hematology, clinical chemistry and circulating caINS-Alb protein will be analyzed regularly. Serum levels of caINS-Alb are measured using a bespoke sandwich ELISA. The requirement for supplementary insulin, insulin dose, body weight and glycemic control as well as improvement in clinical signs will be used to evaluate the efficacy of the test product.

### Example 6: Evaluation of AAV-feINS-Alb Efficacy in a Feline Diabetes Model

A study will be conducted to evaluate the efficacy of a single administration of an AAVrh91 virion expressing feINS-Alb to adequately maintain blood glucose levels in diabetic cats.

Cats with previously diagnosed diabetes mellitus on stable insulin treatment with moderate to good glycemic control will be treated with either placebo or AAV-feINS-Alb (administered intramuscularly). Newly diagnosed cats with diabetes mellitus will be treated with either placebo or AAV-feINS-Alb (administered intramuscularly). Prior to administration of the AAV-feINS-Alb, a series of health screening measures including veterinary examinations, serum fructosamine analysis as well as hematology, clinical chemistry analysis, and urinalysis will be performed. Glycemic control will be assessed prior to administration and throughout the study by measuring interstitial glucose concentrations continuously by a continuous glucose monitor or twice daily using a glucometer. Throughout the study, cats will be fed a consistent diet and body weight will be monitored. Over the course of study, in addition to the blood glucose monitoring, blood collections for fructosamine analysis, hematology, clinical chemistry and circulating feINS-Alb protein will be analyzed regularly. Serum levels of feINS-Alb will be measured using a bespoke sandwich ELISA. The requirement for supplementary insulin, insulin dose, body weight and glycemic control as well as improvement in clinical signs and remission rates and time to remission will be used to evaluate the efficacy of the test product.

To measure serum concentrations of feINS-Alb, a bespoke sandwich ELISA will be used as follows. ELISA plates are coated with 0.2 ug/ml of mouse anti-human insulin (clone 7F8, Life Technologies) and blocked with PBS/0.05% Tween 20/1% casein. Coated wells are incubated for 1 h at room temperature with 100 μL of appropriately diluted serum samples, diluted in PBS/0.05% Tween 20/1% casein. Purified feINS-Alb standards ranging from 200 ng/ml to 0.19 ng/ml are used to establish a standard curve. After washing, the plates are incubated for 1 h with a 1/2000 dilution of goat anti-cat albumin (Bethyl Laboratories) in PBS/0.05% Tween 20/1% casein. Plates are then washed five times with PBS/0.05% Tween 20 and developed by the addition of TMB substrate. Development is stopped by the addition of 2M H₂SO₄ and absorbance is read at 450 nm.

All publications cited in this specification, are incorporated herein by reference. U.S. Provisional Patent Application No. 63/315,252, filed Mar. 1, 2022 is incorporated herein by reference in its entirety. While the invention has been described with reference to particular embodiments, it will be appreciated that modifications can be made without departing from the spirit of the invention. Such modifications are intended to fall within the scope of the appended claims.

1. A recombinant adeno-associated viral (rAAV) virion for treatment of companion animals, comprising an AAV capsid and a vector genome comprising an expression cassette comprising a polynucleotide encoding a fusion protein comprising a proinsulin and serum albumin, the expression cassette flanked by a 5′ inverted terminal repeat (ITR) and a 3′ ITR, wherein the proinsulin is a canine proinsulin or a feline proinsulin.

2. The rAAV virion of claim 1, wherein the AAV capsid comprises AAVrh91 capsid.

3. (canceled)

4. The rAAV virion of claim 1, wherein proinsulin is canine proinsulin and the sequence is at least 80% identical to SEQ ID NO: 14.

5. (canceled)

6. The rAAV virion of claim 1, wherein proinsulin is feline proinsulin and the sequence is at least 80% identical to SEQ ID NO: 15.

7. (canceled)

8. The rAAV virion of claim 1, wherein the fusion protein comprises a polypeptide that shares at least 80% identity the with SEQ ID NO: 1.

9. The rAAV virion of claim 1, wherein the fusion protein comprises an N-terminal signal peptide.

10. The rAAV virion of claim 9, wherein the signal peptide is a canine insulin signal peptide, wherein the signal peptide comprises the sequence that shares at least 80% identity with
(SEQ ID NO: 7)
MALWMRLLPLLALLALWAPAPTRA

11. (canceled)

12. The rAAV virion of claim 1, wherein the canine proinsulin is a canine proinsulin variant having a mutation at one or more cleavage sites compared to a reference polypeptide sequence as set forth in SEQ ID NO: 10.

13. The rAAV virion according to claim 1, wherein the proinsulin is a canine proinsulin fused to a canine serum albumin, and wherein canine proinsulin-serum albumin fusion polynucleotide shares at least 80% identity with SEQ ID NO: 2.

14. (canceled)

15. The rAAV virion of claim 12, wherein the canine proinsulin comprises K29R, R31K, and L62R mutations compared to a reference polypeptide sequence as set forth in SEQ ID NO: 10.

16. The rAAV virion of claim 13, wherein the canine proinsulin-serum albumin fusion protein comprises a linker that shares at least 80%, at least 85%, at least 90%, at least 95%, at least 99%, or 100% identity to SEQ ID NO: 8.

17. (canceled)

18. The rAAV virion of claim 9, wherein the signal peptide is a feline signal peptide and comprises the sequence that shares at least 80% identity with SEQ ID NO: 9.

19. The rAAV virion of claim 1, wherein the feline proinsulin is a feline proinsulin variant having a mutation at one or more cleavage sites compared to a reference polypeptide sequence as set forth in SEQ ID NO: 24.

20. The rAAV virion according to claim 1, wherein the proinsulin is a feline proinsulin fused to a feline serum albumin, and wherein the feline proinsulin-feline serum albumin fusion polynucleotide shares at least 80% identity with SEQ ID NO: 33.

21. (canceled)

22. The rAAV virion of claim 19, wherein the feline proinsulin comprises K29R, R31K, and L62R mutations compared to a reference polypeptide sequence as set forth in SEQ ID NO: 24.

23. The rAAV virion of claim 1, wherein the polynucleotide encoding the fusion protein is operatively linked to a promoter.

24. (canceled)

25. The rAAV virion of claim 1, wherein the expression cassette comprises polynucleotide sequence encoding a homology directed repair (HDR) template configured for insertion into a cut site.

26-28. (canceled)

29. The rAAV virion of claim 1, wherein the virion composition is formulated to be administered to the canine or feline subject at a dose of 1×10⁹ GC/kg to 3×10¹³ GC/kg of the rAAV and/or wherein the rAAV is delivered intramuscularly.

30. A method of treating a canine or feline subject having a metabolic disease, comprising administering to the canine or feline subject an effective amount of the rAAV virion of claim 1 or the pharmaceutical composition of claim 26.

31. The method of claim 30, wherein the metabolic disease is diabetes.

32-39. (canceled)
